(19) United States
(12) Patent Application Publication
(10) Pub. No.: US 2025/0179003 A1
(43) Pub. Date: Jun. 5, 2025

(54) ETHYL ACETATE PRODUCTION AND PURIFICATION

(71) Applicant: VIRIDIS CHEMICAL, LLC, Kingwood, TX (US)

(72) Inventor: Amit HASABNIS, Kingwood, TX (US)

(21) Appl. No.: 18/846,149

(22) PCT Filed: Feb. 15, 2024
(86) PCT No.: PCT/US2024/015982

§ 371 Date: Sep. 11, 2024

Related U.S. Application Data
(60) Provisional application No. 63/485,188, filed on Feb. 15, 2023.

(51) Int. Cl. C07C 67/58; C07C 67/40

(52) U.S. Cl. CPC C07C 67/58; C07C 67/40

(57) ABSTRACT

A method of purifying an ethyl acetate stream includes contacting an inlet stream with a solvent, transferring at least a portion of the impurity compound from the inlet stream into the solvent to form an extract and a purified product, separating the extract from the purified product; separating the portion of the impurity compound from the extract, preventing the formation of foam while separating the portion of the impurity compound from the extract using the defoamer, forming an impurities stream and a regenerated solvent, and recycling at least a portion of the regenerated solvent to contact the inlet stream. The inlet stream comprises ethyl acetate and an impurity compound, and the solvent comprises an extracting agent and a defoamer. The extract comprises the solvent and the portion of the impurity compound transferred from the inlet stream.

### CROSS-REFERENCE TO RELATED APPLICATIONS

The present application claims priority to U.S. Provisional Application No. 63/485,188 filed on Feb. 15, 2023 and entitled, “Ethyl Acetate Production and Purification,” which is incorporated herein by reference in its entirety.

### BACKGROUND

The following discussion is provided solely to assist the understanding of the reader, and does not constitute an admission that any of the information discussed or references cited constitute prior art to the present invention.

Ethyl acetate can be produced from acetaldehyde according to the following reaction:

2CH₃CHO←→CH₃COOC₂H₅

When acetaldehyde is produced from ethanol, ethyl acetate can be produced from ethanol according to the following reaction:

2C₂H₅OH←→CH₃COOC₂H₅+2H₂

Alternately, ethanol can react with acetaldehyde according to the following reaction:

C₂H₅OH+CH₃CHO←→CH₃COOC₂H₅+H₂

One particular problem in production of ethyl acetate by dehydrogenation of ethanol is that the reaction product mixture tends to be a complex mixture including esters, alcohols, aldehydes and ketones. The reaction mixture can be even more complex when the ethanol feed contains impurities. The reaction product mixtures contain components with boiling points close to ethyl acetate (such as N-butyraldehyde and butan-2-one), including components which can form azeotropes with ethyl acetate, and/or other components of the mixture. Another problem is that water present in the feed ethanol or produced as a by-product during dehydrogenation has an inhibiting effect on dehydrogenation catalysts based on the concentration of the water so that any recycle to the dehydrogenation reactor of unconverted ethanol should desirably contain only a low level, if any, of water.

Separation of ethyl acetate from a composition comprising ethyl acetate, ethanol and water (and other impurities) can be accomplished by feeding the composition to a distillation column to obtain a quasi-azeotropic mixture comprising ethyl acetate, ethanol and water, condensing it, separating the condensate into an organic layer and an aqueous layer, returning the organic layer to the column, and recovering ethyl acetate as a bottom product from the column. However, some amount of water and impurities can remain in the mixture.

### SUMMARY

In some embodiments, a method of purifying an ethyl acetate stream comprises contacting an inlet stream with a solvent, transferring at least a portion of the impurity compound from the inlet stream into the solvent to form an extract and a purified product, separating the extract from the purified product; separating the portion of the impurity compound from the extract, preventing the formation of foam while separating the portion of the impurity compound from the extract using the defoamer, forming an impurities stream and a regenerated solvent, and recycling at least a portion of the regenerated solvent to contact the inlet stream. The inlet stream comprises ethyl acetate and an impurity compound, and the solvent comprises an extracting agent and a defoamer. The extract comprises the solvent and the portion of the impurity compound transferred from the inlet stream.

In some embodiments, a system for producing high purity ethyl acetate from ethanol comprises a heater configured to heat an inlet ethanol stream, at least one reactor comprising a dehydrogenation catalyst, an ethanol inlet configured to pass ethanol in the inlet ethanol stream from the heater over the dehydrogenation catalyst to produce a product stream comprising ethyl acetate and impurities, an extraction unit configured to receive the product stream from the at least one reactor, contact a liquid solvent feed stream with the product stream, provide an extract stream comprising a portion of the impurities in the product stream, and provide a purified product stream, and a stripping unit configured to receive the extract stream from the extraction unit, separate the impurities from the extract stream, provide an outlet impurities stream, and provide a regenerated solvent stream back to the extraction unit as at least a portion of the liquid solvent feed stream. The liquid solvent feed stream comprises an extracting agent and a defoamer.

In some embodiments, a method of producing ethyl acetate comprises feeding a feed stream comprising ethanol to one or more reactors, contacting the ethanol with a catalyst in the one or more reactors, producing ethyl acetate and one or more byproducts during the dehydrogenation to produce an effluent stream comprising the ethyl acetate and the one or more byproducts, separating a portion of the one or more byproducts to produce a purified product stream, and separating at least a portion of the byproducts from the purified product stream using one or more distillation columns. The solvent comprises an extracting agent and a defoamer.

These and other features will be more clearly understood from the following detailed description taken in conjunction with the accompanying drawings and claims.

### BRIEF DESCRIPTION OF THE DRAWINGS

For a more complete understanding of the present disclosure and the advantages thereof, reference is now made to the following brief description, taken in connection with the accompanying drawings and detailed description.

FIG. 1 shows a simplified schematic of an ethyl acetate production system according to an embodiment.

FIG. 2 shows a simplified schematic of a dehydration system according to an embodiment.

FIG. 3 shows a simplified schematic of a portion of an ethyl acetate production system according to an embodiment.

FIG. 4 illustrates a schematic flow diagram of a product separation system according to an embodiment.

FIG. 5 illustrates a schematic flow diagram of a product separation system according to another embodiment.

FIG. 6 illustrates a schematic flow diagram of a stripping section according to another embodiment.

FIG. 7 illustrates a schematic co-current extraction flow scheme according to an embodiment.

FIG. 8 illustrates a schematic counter-current extraction flow scheme according to an embodiment.

FIG. 9 illustrates a schematic cross-current extraction flow scheme according to an embodiment.

FIG. 10 illustrates a schematic flow diagram of another product separation system according to another embodiment.

FIG. 11 illustrates a schematic flow diagram of still another product separation system according to an embodiment.

FIG. 12 illustrates another schematic flow diagram of still another product separation system according to an embodiment.

### DETAILED DESCRIPTION

An ethyl acetate production system and process are disclosed herein for producing high purity ethyl acetate from ethanol. This process is beneficial as it provides an improved commercial method of upgrading ethanol to ethyl acetate, a more valuable product. This improved commercial process may be used where there is a supply and/or a surplus supply of ethanol. Further, this process reduces and/or eliminates the need for a separate acetaldehyde or acetic acid plant to provide the precursors for the ethyl acetate production process. The raw material may comprise only ethanol, which may present an advantage relative to other processes requiring multiple feedstocks. In addition, bio-derived ethanol may be used to allow the process to be operated from renewable ethanol sources. Further, the present system and method may utilize base-metal catalysts, which may be less expensive than the precious metal-based catalysts of other ethyl acetate production routes. The present systems and methods may allow for a one-step ethyl acetate production process, which may be advantageous relative to other processes that require further steps to purify the ethyl acetate product, including a selective removal of 2-butanone, which forms a low boiling azeotrope with ethyl acetate. Each of these advantages may be provided in a process that can also be less expensive than alternative processes by ethyl acetate production from ethanol.

In an embodiment, the present systems and methods can provide a route to ethyl acetate by dehydrogenation and dimerization of ethanol which is capable of yielding high purity ethyl acetate from ethanol feed streams containing significant amounts of byproducts or impurities. One issue in the production of ethyl acetate by dehydrogenation of ethanol is that the reaction product mixture is commonly a complex mixture including esters, alcohols, aldehydes and ketones. From a distillative separation point of view, the mixture is further complicated due to the presence of azeotropes. The reaction product mixtures commonly contain components with boiling points close to ethyl acetate (such as n-butyraldehyde and/or butan-2-one), including components which can form azeotropes with ethyl acetate, and/or other components of the mixture.

The present disclosure provides an improved process for the production of high purity ethyl acetate from ethanol, or from a feedstock comprising a major proportion of ethanol and a minor proportion of impurities such as iso-propanol and iso-butanol. While not commonly present in ethanol feed streams, impurities that can poison the particular catalyst used should be limited, avoided and/or removed. For example, sulfur or nitrogen heterocyclic compounds can frequently act as catalyst poisons and, if present, should be removed before introducing the ethanol feed stream to the reactive distillation column. In an embodiment, the ethanol feed may comprise water. The presence of water in the ethanol feed does not severely reduce the performance of the catalysts, which can tolerate up to 5% water by weight in the ethanol. Ethanol conversion is reduced when using an ethanol source with significant water content, but the reaction selectivity increases. The use of an ethanol feed comprising a small amount of water may be advantageous by allowing for the use a potentially less expensive ethanol source in the form of the ethanol/water azeotrope (about 4.4% water by weight). In some aspects, the water can be removed prior to producing ethyl acetate. This can help to reduce the amount of water that is removed in the downstream processing.

Ethyl acetate can be produced from ethanol according to the following reactions:

C₂H₅OH←→CH₃CHO+H₂

CH₃CHO+C₂H₅OH←→CH₃COOC₂H₅+H₂

The Tishchenko reaction may also provide a potential reaction route for the production of ethyl acetate from ethanol:

C₂H₅OH←→CH₃CHO+H₂

2CH₃CHO←→CH₃COOC₂H₅

The resulting product stream from the reactor(s) can comprise ethyl acetate, water, hydrogen, unreacted ethanol, and some amount of byproducts such as n-butyraldehyde and/or butan-2-one. The impurities may leave the initial separation with the ethyl acetate, which can then be further purified to produce high purity ethyl acetate for sale. As described herein, an extraction process can be used to remove impurities such as n-butyraldehyde and/or butan-2-one. However, within the extraction process, the various components can form a foam that can result in a carryover of a portion of the impurities, making the separation only partially effective. In some aspects, chemicals such as a defoamer can be added into the extraction process to control the production of foam and reduce or eliminate the carryover of impurities. This can allow a higher throughput with an improved purity product.

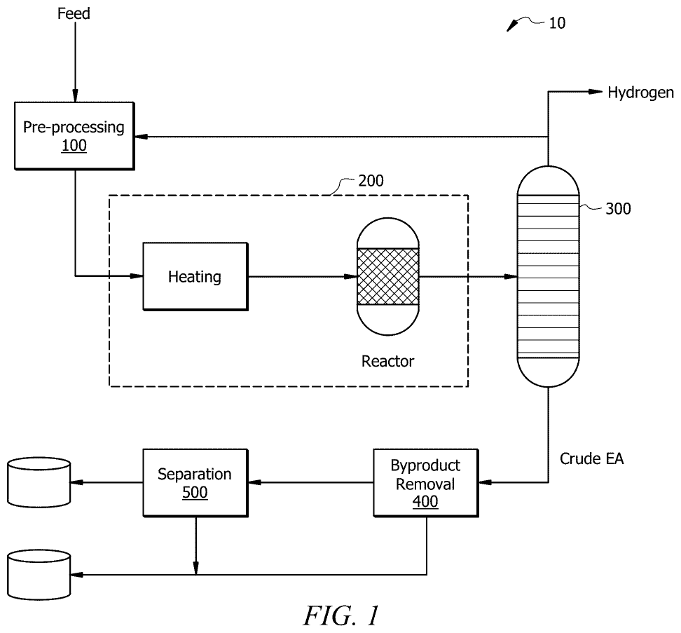

FIG. 1 illustrates a system 10 for producing ethyl acetate. The system can comprise a reactant processing section 100, a reactor section 200, an initial separation and recycle section 300, a byproduct removal section 400, and a product purification section 500. Within the reactant processing section 100, the reactants comprising ethanol can be optionally pre-processed to produce an ethanol feed having a desired composition. In some aspects, various processes such as dehydration, purification, and the like can be used to produce a feed stream having an ethanol purity suitable for use in the reactor section 200. Within the reactor section 200, ethyl acetate can be produced based on contacting the ethanol with a catalyst. Various byproducts and/or impurities can also be generated within the reactor section 200. The outlet stream from the reactor section 200 can then pass to the initial separation section 300 where any unreacted ethanol can be separated and returned to either the reactant processing section 100 and/or the reactor section 200. The remaining crude ethyl acetate can pass to the byproduct removal section 400, where at least some byproducts such as N-butyraldehyde and/or butan-2-one can be removed from the ethyl acetate stream prior to the remaining ethyl acetate passing to the product purification section 500. Within the product purification section 500, one or more separation processes can be used to purify the ethyl acetate to a desired purity. Each of these sections is described in more detail herein.

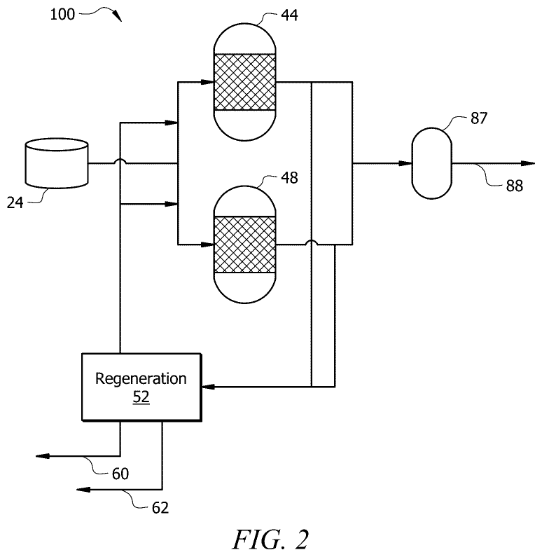

As shown in FIG. 2, the system can comprise a reactant processing section 100 configured to provide a stream comprising ethanol suitable for the reactor section 200. In some embodiments, the reactant processing section 100 can comprise a feed storage 24 and a dehydration system to produce a feed stream with ethanol having a desired purity. As shown, the reactant processing section 100 can comprise one or more storage units such as holding tank 24 suitable for storing an amount of ethanol, which can be delivered using any suitable route such as a pipeline, railcar, shipload, truckload, or the like. While one holding tank is shown, any suitable number of holding tanks can be present in the system.

The ethanol in the holding tank 24 can be in fluid communication with a dehydration system configured to produce an ethanol stream having a water content below a desired concentration. The dehydration system can comprise any suitable units such as distillation units (e.g., extractive distillation, etc.), absorption units, adsorbent beds, extraction units, or the like. In some embodiments, one or more adsorbent beds can be used to dehydrate the ethanol stream. For example, dehydration system may have one or more swing bed adsorption units, such as a first swing bed unit 44 and a second swing bed unit 48 arranged in parallel configuration. The ethanol feed can pass into a first feed line or a second feed line to communicate with respective first swing bed unit 44 or the second swing bed unit 48. Each swing bed unit 44 and 48 can contain an adsorbent, such as a molecular sieve adsorbent (e.g., a zeolite, etc.). An outlet line can be coupled to each swing bed unit 44, 48 to pass the ethanol to the downstream reactor section 200. The ethanol passing out of the swing beds can have a water content of less than about 0.5 wt. %, less than about 0.4 wt. %, less than about 0.3 wt. %, or less than about 0.2 wt. %.

In operation, one swing bed reactor can be adsorbing water from the feed passing therethrough while the other swing bed reactor is being regenerated (e.g., dehydrated). When the bed is saturated, the beds can be reversed so that the regenerated bed can be used to adsorb water while the other bed is regenerated. When a bed is isolated for regeneration, the ethanol can be drained from the bed and passed back to the storage tanks for use within the system. A regeneration unit 52 can provide an inert gas, such as nitrogen or carbon dioxide that may be heated, via an inlet line branching to each respective swing bed reactors 44 and 48 to pass through the non-operating bed, and after passing through the bed, be communicated by an outlet line merging to form a single line returning the regeneration gas back to the regeneration unit 52 for purification and optionally reuse and recovery of any ethanol and water to be transferred to any suitable utility via a wet ethanol line 60 and a desorbed water line 62. Turning back to the ethanol passing through the adsorber, once dried, the ethanol can exit and pass to an optional filter 87 for removing any particles, such as adsorbent particles. Due to the operation being alternated between swing bed reactors 44 and 48, the dried feed can optionally pass into a storage tank or surge drum to provide a steady, dried reactor feed 88 to the reactor section.

Within the reactor section, at least a portion of the ethanol in the feed can be converted to ethyl acetate. The reactions can occur in the presence of a catalyst to produce a reaction mixture. Any suitable reactor configurations can be used such as one or more reactors arranged in series and/or parallel. The reactors can comprise fixed bed reactors, reactive distillation units, fluidized bed reactors, or any other reactors configured to contact the ethanol with the catalyst under conditions suitable for carrying the formation of ethyl acetate.

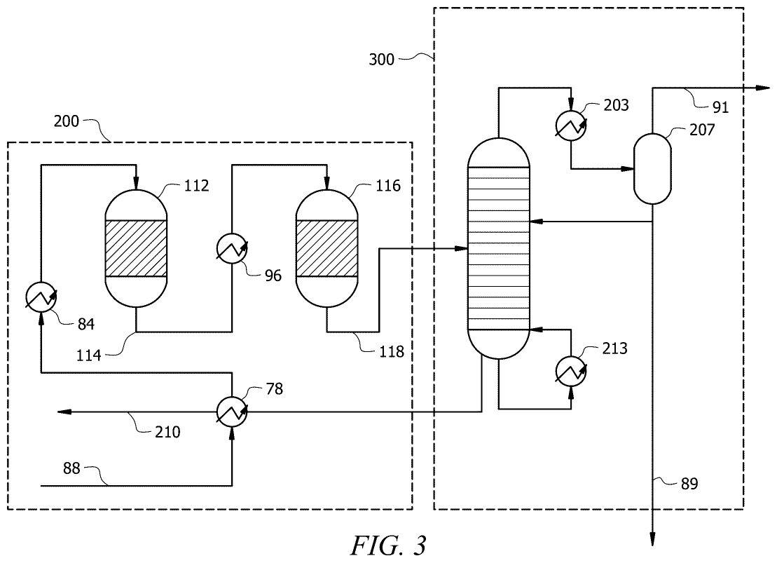

FIG. 3 illustrates an embodiment of a reactor system 200 that can be used for the conversion of at least a portion of the ethanol in the feed stream to ethyl acetate. As shown, the reactor feed 88 can optionally pass through one or more pre-heating exchangers 78 before reacting in one or more reactors 112, 116. The preheat exchanger(s) 78 can include at least one preheat exchanger using any suitable heat source (e.g., combustion, flue gas, heating fluid, one or more hot process streams, etc.). As shown the bottoms stream from separator 201 can be used as the heating fluid to pre-heat the incoming ethanol feed stream while cooling the bottoms stream.

One or more additional heaters can be used to further heat the ethanol to a reaction temperature. In some aspects, a heater 84 can be used to convert the ethanol from a liquid to a vapor (e.g., as a vaporizer) to allow the ethanol to react in the vapor phase. An optional further trim heater can be used to heat the vapor to a desired reaction temperature before the vapor phase ethanol passes to a first reactor 112 comprising the catalyst disposed therein. An additional heater 96 can be arranged in series with one or more additional reactors such as reactor 116 to form a series of heater/exchanger pairs. As the dehydrogenation reaction is typically endothermic, heaters 84, 96 can be positioned, respectively, before a first reactor 112 and a second reactor 116. The first reactor 112 and the second reactor 116 may be, independently, any of the types of reactors as described herein. In some aspects, the reactors can comprise fixed bed adiabatic reactors. As such, a first reactor effluent 114 from the first reactor 112 can be provided to the second reactor 116, and a second reactor effluent 118 from the second reactor 116 can include ethyl acetate, unreacted ethanol, hydrogen, and reaction byproducts.

Any suitable reaction conditions can be used to carry out the ethyl acetate production reactions. In some aspects, the ethanol can be in the vapor phase and can be heated to a temperature of between about 200° C. to about 300° C. and to a pressure from about 1 to about 50 bar upon entry into the first reactor 112. The effluent stream 114 from the first reactor 112 can leave the first reactor at a lower temperature such as between about 10° C. to about 30° C. lower than the inlet temperature of the first reactor, and at a slight pressure drop. The second heater 96 can then serve to reheat the effluent 114 from the first reactor 112 to the reaction temperature of between about 200° C. to about 300° C. While two reactors are shown in series, one reactor or three or more reactors can be used in series with an inlet heater positioned upstream of each reactor. The stream leaving the second reactor 116 can then pass to a separator 201 for an initial separation to allow the unreacted ethanol to be returned to the inlet of the process. The catalyst(s) used herein are described in more detail below.

The product mixture passing from the second reactor 116 to the separator 201 can comprises hydrogen, ethyl acetate, unconverted ethanol, water and minor amounts of byproducts or impurities present either from contamination in the feed, recycle streams or from side reactions in reactors 112, 116. Examples of these impurities include aldehydes, such as acetaldehyde, n-butyraldehyde, and/or crotonaldehyde; ethers, such as ethyl ether and n-butyl ether; other acetates such as butyl acetate or methyl acetate; ketones such as 2-butanone, acetone; and other alcohols, such as isobutanol, 2-butanol, 2-ethylbutanol, n-hexanol, and/or 2-ethylhexanol. Some of the components can comprise byproducts whose boiling points are close to that of ethyl acetate or which form azeotropic mixtures with ethyl acetate. These can include, but are not limited to, certain carbonyl-containing compounds such as acetone, acetaldehyde, and butan-2-one. In some aspects, the product mixture passing through the reactor train to the separator 201 can comprise between about 20 wt. % to about 50 wt. % ethyl acetate, or between about 30 wt. % to about 45 wt. % ethyl acetate, and between about 40 wt. % to about 80 wt. %, or between about 45 wt. % and about 60 wt. % ethanol. Additional components of the product blend can include between about 0.05 to about 4 wt. % of one or more byproducts including, but not limited to, acetaldehyde, acetic acid, acetone, butan-1-ol, butan-2-ol, butyraldehyde, diethylether, ethane, ethene, water, ethylbutyrate, hexan-1-ol, isobutylacetate, methanol, methylacetate, n-butylacetate, pentan-2-ol, propan-2-ol, and/or sec-butylacetate.

An initial separation can be performed in the initial separation and recycle section 300 as shown in FIG. 3. As illustrated the initial separation and recycle section 300 can comprise a separator 201, which can be configured to produce a hydrogen stream 91, an ethanol recycle stream 89, and a crude product stream 210 that can be separated to produce a final ethyl acetate product. In some aspects, the separator 201 can comprise a distillation column. The distillation column may comprise about 1 to about 100 stages, or about 1 to about 50 stages. The distillation column may operate at a pressure ranging from about 1 atm to about 80 atm. The temperature and pressure may be selected based on the equilibrium on each stage within the distillation column. In an embodiment, the temperature within the column may range from about 50° C. to about 250° C., or from about 70° C. to about 200° C.

The separator 201 can produce an overhead stream that can pass to a cooler 203. In some aspects, the cooler 203 can comprise an air cooler or ambient cooler. The overheat stream can be cooled to between about 25-50° C. in the cooler 203 before the stream passes to the knockout drum 207. Within the knock out drum, any components that condense can be removed as a liquid stream, and the remaining gas phase components, which can be a hydrogen rich gas stream 91, can pass out of the system. The hydrogen rich gas phase stream 91 can be sold and/or used as fuel within the system. The liquid from the knockout drum 207 can be split with a first portion passing back to the separator 201 as a reflux stream, and a second portion 89 passing to an inlet of the process as the ethanol recycle stream. The liquid phase from the knockout drum 207 can comprise predominantly unreacted ethanol with some amount of water produced within the system. The recycled ethanol in stream 89 can then pass to a point upstream of the processing section 100 so that any water present can be removed.

A bottoms stream can be removed from the separator 201 and pass to a reboiler 213 to generate a gas phase stream for reflux within the separator 201. A portion of the stream passing to the reboiler 213 and/or a separate bottoms stream can pass out of the separator 201 as the crude ethyl acetate product stream 210. The byproducts, including those that form azeotropes and/or have similar boiling points can be present in the crude product stream 210 passing out of the separator 201 as the bottoms stream.

The crude product stream 210 can then pass to a byproduct removal section 400 for use in removing a portion of any byproducts or impurities from the product stream. In some embodiments, the ethyl acetate product stream produced in the processes and systems described herein may comprise one or more impurities. In some embodiments, the ethyl acetate product stream produced in the reactor section 200 may comprise less than about 15 wt. %, less than about 10 wt. %, less than about 8 wt. %, less than about 6 wt. %, less than about 4 wt. %, less than about 2 wt. %, or less than about 1.5 wt. % byproducts, and in some embodiments, more than about 0.01 wt. %, more than about 0.1 wt. %, more than about 0.5 wt. %, or more than about 1 wt. % byproducts.

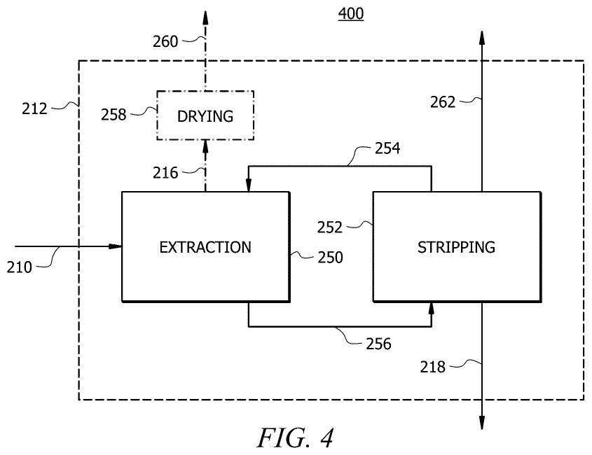

In some aspects, the byproduct removal section 400 can comprise an extraction process for removing a portion of the impurities in the crude product stream 210. As shown in FIG. 4, the crude product stream 210 can be the crude product stream from the initial separation and recycle section 300. In some aspects, the byproduct removal section 400 can also be used with any ethyl acetate stream produced using any process to remove byproducts. For example, an extraction process 400 may be used to separate at least a portion of one or more byproducts from the ethyl acetate in the crude product stream 210, thereby improving the quality and value of the ethyl acetate product stream. The byproduct removal section 400 generally comprises an extraction section 250 in which the crude product stream 210 is contacted with a solvent in a solvent stream 254 to transfer or remove at least a portion of the impurities from the product. The extract stream 256 comprising the impurities transferred from the product can then be regenerated in a stripping section 252, which generally involves separating the impurities from the solvent to allow the impurities to be removed from the system and the solvent to be recycled.

The extraction process 400 may comprise the extraction section 250 in which the crude product stream 210 is contacted with a solvent in a solvent stream 254. In an embodiment, the extraction section 250 comprises a liquid-liquid contact vessel suitable for contacting two liquid streams. Suitable vessels and extraction section 250 configurations are described in more detail herein and can include, but are not limited to, sieve tray columns, packed columns, pulsed columns, rotating disc contactor (RDC) columns, SCHEIBEL columns (e.g., a rotating impeller column), and KARR columns (e.g., an agitated plate column), or the like. The solvent may draw a portion of one or more impurities from the crude product stream 210 into the solvent phase. The crude product stream 210 having the portion of the impurities removed may then exit the extraction section 250 as a purified product stream 216. The extract comprising the solvent and the portion of the impurity compound transferred from the crude product stream 210 may then exit the extraction section 250 as an extract stream 256.

An optional drying section 258 may serve to remove any remaining solvent or a component of a solvent (e.g., water from an aqueous solvent) from the purified product stream 216. The resulting dried purified product stream 260 may then leave the byproduct removal section 400. Any suitable drying units may be used to remove at least a portion of the solvent from the purified product stream 216. Suitable drying units may include, but are not limited to, industrial dehydration units comprising adsorbents such as Zeolites, alumina, silica, and other drying agents arranged in a pressure and/or temperature swing configuration, and/or liquid absorption (e.g., liquid-liquid extraction, gas-liquid extraction, etc.) using a drying agent. The purified product stream 216 and/or the dried purified product stream 260 may comprise ethyl acetate having a purity of greater than about 90%, greater than about 95%, greater than about 96%, greater than about 97%, greater than about 98%, greater than about 99%, or greater than about 99.5% ethyl acetate by weight.

In order to separate the impurities from the solvent, the extract stream 256 may be transferred to a stripping section 252. In an embodiment, the impurities may be removed using vapor-liquid stripping, heating, liquid-liquid phase separation, flashing and/or distillation, and/or any other suitable technique. Suitable vessels and stripping section 252 configurations are described in more detail herein. As shown in FIG. 4, the stripping section 252 may result in the separation of the impurities from the extract stream 256, thereby producing a liquid impurities stream 218 and/or a vapor impurities stream 262, and a regenerate solvent stream 254. In some embodiments, different components of the impurities may leave the separation section 212 as either a vapor or a liquid, including those generated by the solvent and/or any reactions within the separation section 212. The solvent may be regenerated by removing at least a portion of the impurities in the stripping section 252. In some embodiments, only a portion of any extracted impurities can be removed in the stripping section 252, and the resulting regenerated solvent stream 254 may comprise a minor amount of impurities. The regenerated solvent stream 254 may be recycled to the extraction section 250 to serve as the solvent. In some embodiments, a new solvent stream (e.g., a makeup solvent stream) may enter the extraction section separately or with the regenerated solvent stream 254 to serve as the solvent in the extraction section 250. The liquid impurities stream 218 and/or vapor impurities stream 262 may exit the system for various downstream uses. In an embodiment, the impurities stream may comprise commercially valuable chemicals that can be transferred for sale. In some embodiments, the impurities may be further separated before being sold, or alternatively, the impurities can be used as fuel or any other suitable use.

When used with the ethyl acetate production systems and processes describe herein, the solvent used in the extraction process may be selected to remove the expected impurities within the ethyl acetate product stream (e.g., crude product stream 210). As noted above, the synthesis of ethyl acetate by consecutive dehydrogenation and condensation of ethanol to ethyl acetate is accompanied by the formation of relatively small amounts of byproducts. At least a portion of the byproducts comprising various aldehydes, ketones, and the like can be removed to improve the quality and purity of the ethyl acetate. In an embodiment, the extraction process can be used to selectively extract at least a portion of the byproducts that may be present in the ethyl acetate. In an embodiment, the solvent may be configured to selectively react with and extract the impurities in the ethyl acetate. For example, the solvent may comprise one or compounds configured to reversibly react with the impurities, thereby allowing the reaction to remove the impurities from the ethyl acetate in the extraction section 250 and then be reversed to release the impurities in the stripping section 252.

In an embodiment, the solvent may comprise a fluid comprising one or more extracting agents. In an embodiment, the extracting agent may comprise a nucleophilic compound that can react with aldehydes and/or ketones to form a corresponding adduct. The resulting reaction products may have an increased solubility in the aqueous fluid, thereby allowing the adducts to leave the crude product stream 210 and enter the solvent stream 254 to form the extract stream 256. Within the stripping section 252, the adduct equilibrium may be shifted to release the extracting agents from the impurities. The impurities may then be separated from the solvent comprising the extracting agents using any suitable separation technique such as vaporization and/or liquid-liquid separation.

Various extracting agents can be used in the aqueous solvent. In an embodiment, the extracting agents can comprise sulfur and/or nitrogen. Suitable extracting agents can include, but are not limited to, hydrazine (N₂H₄), hydroxylamine (NH₂OH), semicarbazide (H₂NNH(C═O)NH₂), phenylhydrazine (C₆H₅N₂H₃), phenylhydroxylamine (C₆H₅NHOH), sodium hydrogen sulfite (NaHSO₃/Na₂S₂O₅), sodium sulfite (Na₂SO₃), salts thereof, aqueous solutions thereof, and any combinations thereof. While any of these extracting agents can be used, reference will be made to sodium hydrogen sulfite in the following discussion as an example.

In an embodiment, an aqueous solution of sodium hydrogen sulfite (which can also be referred to as sodium bisulfite) can be used as the solvent in the extraction process. Sodium hydrogen sulfite can be sourced as a solid, which usually comprises a mixture of NaHSO₃ and Na₂S₂O₅. An aqueous solution can be made from the salt, and the use of an aqueous solution of sodium hydrogen sulfite provides an inexpensive, low toxicity solution that can serve as the solvent in the extraction process. Additional extracting agents can include sodium sulfite (Na₂SO₃). Sodium sulfite can be used by directly dissolving sodium sulfite in a solvent, or sodium hydroxide (NaOH) can be added to sodium hydrogen sulfite as a solid or in solution. Further a mixture of sodium hydrogen sulfite and sodium sulfite can be used in any ratio. The reactive moiety in the aqueous solution of sodium hydrogen sulfite is the hydrosulfite ion (HSO₃⁻/S₂O₅²⁻) and reactive moiety in an aqueous solution of sodium sulfite is the sulfite ion (SO₃²⁻). Thus, any soluble hydrosulfite and/or sulfite compound can also be used in similar manner regardless of the counter ion. For example, hydrosulfite compounds including K⁺, NH₄⁺ salts can also be used along with or in place of sodium hydrogen sulfite.

The aqueous solution comprising the hydrosulfite ion reacts reversibly with aldehydes and ketones such as 2-butanone, acetone, acetaldehyde, thereby rendering them more water soluble as sodium salts of hydrosulfite adducts of the corresponding compounds. For example, the following reaction may take place when the aqueous solution comprising the hydrosulfite ion is contacted with a fluid comprising a ketone (e.g., 2-butanone) as follows:

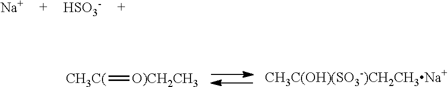

The resulting sodium salt of the 2-butanone sulfonate adduct has higher solubility in water compared to ethyl acetate thus leading to partial extraction of 2-butanone from the organic (ethyl acetate) phase, thereby increasing the ethyl acetate phase purity. The resulting adduct may be formed by various aldehydes and/or ketones, thereby providing for the ability to selectively improve the removal efficiency of the impurities generated in the ethyl acetate production process during the extraction section 250.

Within the extraction process, various factors may affect the extraction efficiency of the solvent within the extraction section 250 including the concentration of the extracting agent in the solvent, the ratio of the solvent to the ethyl acetate product, and the temperature at which the extraction takes place. With respect to the concentration of the extracting agent in the solvent, there are several considerations that can have an effect on the concentration of the compound in the solvent. In general, a higher concentration of the extracting agent (e.g., NaHSO₃) in the aqueous fluid can reduces the ethyl acetate solubility in the solution. However, as the concentration of the extracting agent rises above a certain amount, the resulting adduct may precipitate out of solution. Such precipitation may complicate a continuous process in terms of solids handling equipment. In general, the amount of the extracting agent in the aqueous fluid should be sufficient to provide an excess of the ions in solution but less than an amount that would result in precipitation of the resulting adduct in the extract, which indicates that the solubility limit of the adduct has been reached. Thus, the concentration of the extracting agent in the solvent may vary based on the concentration of the impurities in the ethyl acetate product stream. In an embodiment, the concentration of the extracting agent (e.g., sodium hydrogen sulfite, etc.) in the aqueous solution can vary. In an embodiment, the extracting agent can comprise sodium hydrogen sulfite, and the amount of sodium hydrogen sulfite in the aqueous solution can vary and may generally range from about 50 grams to about 420 grams, from about 300 grams to about 420 grams of solid (e.g., a mixture of NaHSO₃ and Na₂S₂O₅) per liter of water. When the extracting agent is sodium hydrogen sulfite, the concentration may generally range from about 5 to about 42 grams per 100 milliliters of solution, or from about 30 to about 42 grams per 100 milliliters of solution at about 68° F. (about 20° C.). In an embodiment, the extracting agent may be provided in the form of sodium sulfite, a hydrate thereof, and/or SO₂, and the concentration may generally range from about 5 to about 68 grams per 100 milliliters of solution when the sodium sulfite is provided as sodium sulfite heptahydrate. The concentration of the extracting agent may vary based on the composition of the extracting agent, the form in which it is provided, the temperature of the solution, the composition of the solution, and the like. Makeup extracting agent can be added at any suitable location in the system. For example, the makeup extracting agent can be added into the extract stream 256, the stripping section 504, and/or the solvent stream 319.

With respect to the temperature at which the extraction process takes place, the extraction efficiency of the impurities in the ethyl acetate with the aqueous solvent generally increases at a lower temperature relative to a higher temperature extraction. Accordingly, lowering the temperature of the extraction process may result in a greater amount of the impurities being transferred from the ethyl acetate product to the solvent stream. In general, the extraction section 250 may operate within a temperature of between about 0° C. to about 70° C., from about 5° C. to about 60° C., or from about 10° C. to about 50° C.

The solvent can also comprise a defoamer. It has been discovered that the extraction process can result in the formation of a foam that can result in the carryover of the solvent and byproducts into the purified product stream. The foam can be generated using any of the liquid-liquid contactors described herein including those using agitation or other methods to increase the interfacial contact between the solvent phase and the organic phase (e.g., the ethyl acetate phase). In order to help reduce or eliminate the formation of foam, the solvent can comprise any suitable defoamer. The defoamer can be retained within the aqueous phase during the extraction and the stripping sections. Any amount of defoamer that is lost to the purified product stream 260 and/or a byproduct stream 218, 262 can be made up in a make up stream introduced into the solvent, for example, the solvent stream passing between the stripping section and the extraction section.

In some aspects, the defoamer can comprise a primary alcohol component and an alcohol alkoxylate component. The primary alcohol component comprises at least one linear primary alcohol, and/or mixtures of primary linear alcohols. For example, the linear primary alcohols can comprise C₁₄ to C₃₂ primary linear alcohols (e.g., fatty alcohols), or alternatively C₁₈ to C₃₂ primary linear alcohols. The alcohol alkoxylate component can comprise one or more alcohol alkoxylates which function with the primary alcohol component to enable defoaming and/or the inhibition of the formation of foam. The alcohol alkoxylates can include combinations of alkoxylates that add both hydrophobic and hydrophilic groups to the alcohol alkoxylate. For example, the alcohol alkoxylates can include ethylene oxide groups, which comprise the hydrophilic groups, and propylene oxide groups, which comprise the hydrophobic groups. The alcohol alkoxylate can also contain a linear or branched alkyl chain containing 12 to 18 carbons. This alkyl chain is also a hydrophobic group. The alcohol alkoxylate can have a larger proportion of hydrophobic groups, such as but not limited to the propylene oxide groups, so that the alcohol alkoxylate can be considered to have an overall hydrophobic characteristic even though the alcohol alkoxylate contains hydrophilic and hydrophobic groups.

The defoamer can also comprise an emulsifying agent. The emulsifying agent component can comprise one or more of any emulsifier(s) capable of dispersing the primary alcohol component and the alcohol alkoxylate into aqueous media. The at least one emulsifying agent component can be present at any concentration that enables the dispersion of the components into the aqueous media. For example, the emulsifying agent can comprise at least one nonionic surfactant. The nonionic surfactant that can be used in the emulsifying agent component of the present invention can include, but is not limited to, alcohol ethoxylates, fatty acid ethoxylates, alkyl phenol ethoxylates, sorbitan esters, sorbitan ester ethoxylates, ethylene oxide/propylene oxide copolymers, glycol esters, glyceryl esters, polyglycerides and polyoxyalkylene glyceride esters. The emulsifying agent component of the present invention can also include anionic surfactants which include, but are not limited to, alcohol sulfates; alkylaryl sulfonates; alkyl benzene sulfonates: ethoxylated alcohol sulfates; sulfates and sulfonates of ethoxylated alkyl phenols; sulfates of fatty esters; sulfates and sulfonates of alkyl phenols; sulfonates of condensed naphthalenes; sulfonates of naphthalene; sodium derivatives of sulfo-succinates; alkali salts of petroleum sulfonates; alkali phosphate esters and the like. In some aspects, the concentration of the emulsifying agent can range from about 0.2 to 5.0 weight percent, or the concentration of the emulsifying agent component can range from about 0.5 to 3 weight percent.

The defoamer composition can includes from about 10 to about 35 weight percent primary alcohol, from about 2 to about 12 weight percent alcohol alkoxylate, and from about 0.2 to about 5 weight percent of at least one emulsifying agent component, and the balance being water. In some aspects, additional additives can be present such as additives conventionally incorporated into defoaming compositions. The additives can include branched alcohols, stabilizing and/or thickening agents, preservatives, and the like. Suitable defoamers can include those available from Suez WTS USA, Inc. of Trevose, PA under the trade name FOODPRO FAF9814. The defoamer can be added to the solvent in an amount of between about 0.5 to about 2 wt. % of the total solvent composition.

In some embodiments, the defoamer composition can comprise a silicon based defoamer composition. The silicone based defoamer can comprise linear or branched silicones terminated by polymethylsilyl groups such as polydimethylsiloxanes. This group of defoamers can more broadly be described as comprising organopolysiloxanes. Additional components can also be included in the defoamer such as silica, paraffinic oils, low molecular weight polyolefin wax dispersions, long chain alcohols and their esters; fatty acids; silicone fluids, including both trimethylsilyl-terminated fluids and dimethylsilanol-terminated fluids; and/or silicone resins, particularly low molecular weight liquid silicone resins.

Once the aqueous solvent contacts the ethyl acetate product stream (e.g., crude product stream 210) in the extraction section 250, the aqueous fluid comprising the adducts can be separated from the organic ethyl acetate phase using liquid-liquid separation. Foam can be reduced within the extraction process (e.g., in the extraction process or stripping process) based on using the defoamer. The separated extract stream 256 can then be used to perform an additional extraction of impurities from an ethyl acetate product stream and/or the extract can be transferred to the stripping section 252 for regeneration. In general, the extract stream 256 comprising the extracted byproducts (e.g., as adducts or solvated components) can be treated in the stripping section 252 to separate the organic compounds (e.g., the impurities, any dissolved ethyl acetate, etc.) from the aqueous solvent comprising the extracting agent. For example, the extract stream may be heated to reverse the adduct formation, and the organic phase can then be separated from the aqueous solution. In some embodiments, the adducts may be separated by crystallization followed by a subsequent treatment of the precipitated solids.

When the adducts remains dissolved in the aqueous fluid, the adducts can be decomposed by heating to reverse the equilibrium and release the impurities (e.g., the aldehydes/ketones) while regenerating the aqueous solution comprising the extracting agent for reuse. The impurities can then be separated using evaporation, distillation, and/or liquid-liquid phase separation. In an embodiment, the aqueous solution (e.g., the extract stream 256) may comprise the excess of sodium hydrogen sulfite, the dissolved sodium hydrogen sulfite adducts, and some amount dissolved ethyl acetate. The solution can be regenerated by heating the solution to a temperature suitable to reverse the adduct formation equilibrium. In an embodiment in which the nucleophile compound comprises sodium hydrogen sulfite, the regeneration can be achieved by heating the solution to a temperature between about 45° C. and about 100° C. At this temperature, the sodium hydrogen sulfite adduct decomposes to release the impurities and the NaHSO₃. The released impurities and any dissolved ethyl acetate can be separated from the aqueous solution and collected. In some embodiments, the organic compounds can be vaporized (e.g., in a flash tank) or distilled from the aqueous solution, and/or alternatively the organic compounds can be separated based on a liquid-liquid phase separation. The remaining aqueous solution may comprise the regenerated solvent for use in the extraction process. In an embodiment, the separation process within the stripping section 252 may result in some minor amount of the organic compounds remaining in the aqueous solvent.

During the heating process some amount of the extracting agent may decompose to form an additional impurity. For example, when the extracting agent comprises sodium hydrogen sulfite, heating may result in some sulfur dioxide being lost due to decomposition. The decomposition products can be captured using an aqueous NaHSO₃ solution or an aqueous caustic solution (e.g., NaOH), which may be advantageous in preventing the release of the decomposition products to the atmosphere. The regenerated solvent solution in the stream 254 may have higher or lower extracting agent and/or defoamer concentration than desired. The concentration of one or more components may be adjusted through the addition of water, solid extracting agent, or an extracting agent solution until the desired concentration is reached.

In some embodiments, the adducts can be removed from the extract stream 256 by crystallization. For example, the adducts can be separated from the extract stream 256 by forming a sodium ketone/aldehyde hydrosulfite adduct crystals. If the adducts are crystalized out of solution, the resulting crystals can be separated physically. The crystals can then be heated to reverse the adduct formation. In an embodiment, the crystals may be dissolved in an aqueous solution prior to heating to reverse the adduct formation. The resulting organic impurities can then be separated from the extracting agent and any aqueous solution.

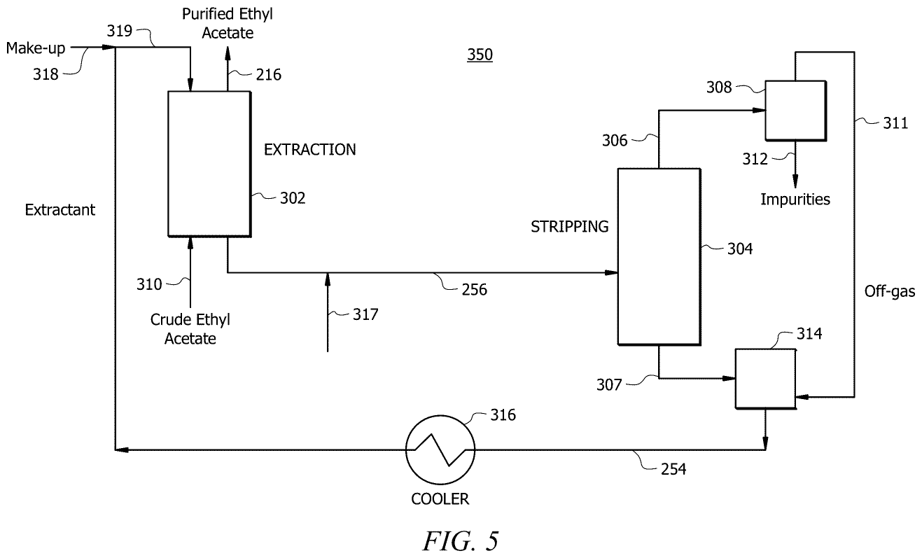

The extraction process can be carried out using various configurations and designs. An embodiment of an extraction process 350 is schematically illustrated in FIG. 5. In this embodiment, a product stream 310 is fed to the extraction section 302. The product stream may generally correspond to the crude product stream 210 or any other stream comprising ethyl acetate and one or more impurities. The product stream 310 may predominantly comprise ethyl acetate, though various byproducts including ketones and/or aldehydes such as 2-butanone, acetone, and/or acetaldehyde may also be present.

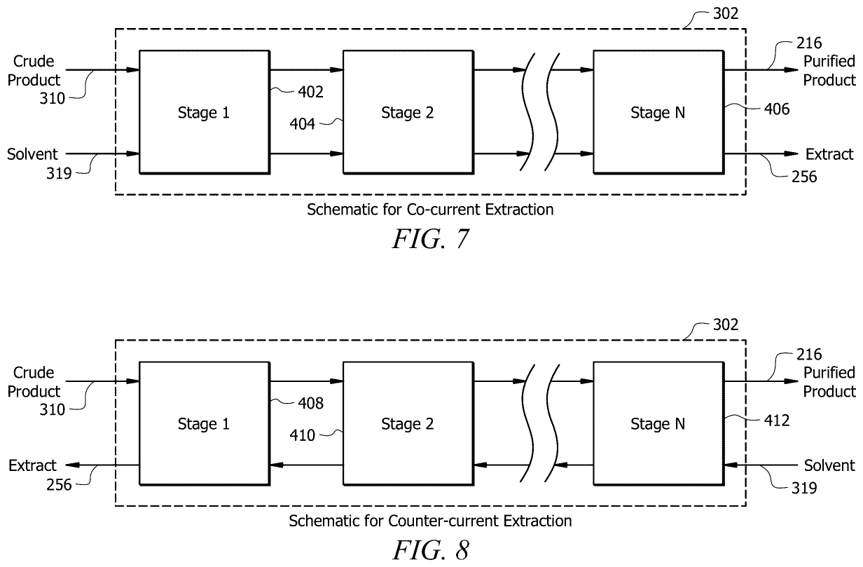

The extraction section 302 may also receive a solvent stream 319. In an embodiment, the solvent stream 319 may comprise an aqueous fluid, an extracting agent, and a defoamer as described above. The extracting agent in the solvent stream may comprise an aqueous solution of hydrazine (N₂H₄), hydroxylamine (NH₂OH), semicarbazide (H₂NNH(C═O)NH₂), phenylhydrazine (C₆H₅N₂H₃), phenylhydroxylamine (C₆H₅NHOH), sodium hydrogen sulfite (NaHSO₃/Na₂S₂O₅), sodium sulfite (Na₂SO₃), salts thereof, aqueous solutions thereof, and any combination thereof. Additional solvents useful with the extraction process can include dichloromethane-ethanol and methyl isobutyl ketone (MIBK). The defoamer in the solvent stream can comprise any of the components as described herein. The extraction section 302 may comprise one or more liquid-liquid contact devices configured to contact the two liquid streams 319, 310. Various liquid-liquid contact devices can be used including an extraction column, which may comprise packing material (e.g., structured packing, random packing, trays, etc.) configured to increase mixing and the available contact area for mass transfer between the two liquids. Additional suitable structures can include a series of mixer settlers, sieve trays, a Kerr-McGee extractor, a packed tower, a rotating disk contactor, a Scheibel extractor, a Karr extractor, a pulsed column, and a centrifugal extractor. The extraction may be carried out in a co-current, counter-current, and/or cross-current flow scheme, each of which is described in more detail below with respect to FIGS. 7, 8, and 9, respectively. In an embodiment, the extraction section can comprises a counter-current flow arrangement between the solvent and product streams.

A contact device within the extraction section may comprise a number of stages configured to provide the degree of impurity removal from the product stream 310 and/or a desired purity of the purified ethyl acetate product stream. In an embodiment, the contact device, or a plurality of contact devices, can contain between about 1 to about 100 stages, or between about 1 to about 50 stages. The contact device or contact devices may operate at any suitable pressure. In an embodiment, the contact device may operate at a pressure ranging from about 1 atm to about 80 atm, and a temperature of between about 10° C. to about 70° C., from about 15° C. to about 60° C., or from about 20° C. to about 50° C.

Within the extraction section 302, at least a portion of the impurities may be transferred to the solvent. In an embodiment, the extracting agent may react with a portion of the impurities in the product stream 310 to form an adduct and improve the solubility of the impurity in the solvent phase. Foam resulting from one or more components of the resulting mixture can be controlled, reduced, or prevented due to the presence of the defoamer in the solvent. Once the product stream 310 and the solvent stream 319 have been contacted and separated within the extraction section 302, a purified product stream 216 and an extract stream 256 may exit the extraction section 302. The purified product stream 216 may have an increased purity of ethyl acetate relative to the entering product stream 310. As described above, the purified product stream 216 may pass to a drying unit to further remove any solvent to further purify the purified product stream. In an embodiment, the purified product stream 216 and/or a dried, purified product stream may comprise ethyl acetate having a purity of greater than about 90%, greater than about 95%, greater than about 96%, greater than about 97%, greater than about 98%, greater than about 99%, or greater than about 99.5% ethyl acetate by weight.

The extract stream 256 may comprise the solvent and the impurities removed from the product stream 310. The extract stream 256 may pass to the stripping section 304. While the defoamer can be added at any point in the circulation of the solvent between the extraction and stripping sections, the defoamer in stream 317 can be added in some embodiments in the extract stream 256 between the extraction and stripping sections. In an embodiment, the extract stream 256 may comprise a solvent fluid, the adducts formed by the reaction between the extracting agent and the impurities, any excess extracting agent, the defoamer, and potentially some amount of dissolved ethyl acetate. The stripping section 304 may comprise one or more separation devices configured to separate the impurities from the solvent and any extracting agent. Various separation devices can be used including phase separators and liquid-liquid separators. In an embodiment, the separation device within the stripping section 304 may comprise a flash tank or a separation column such as a packed column. In this embodiment, the extract stream 256 may enter the flash tank, be heated to a temperature sufficient to dissociate the adduct, and evaporate at least a portion of any impurities and dissolved ethyl acetate. When a packed column is used, the extract stream 256 may enter an upper portion of the packed column and pass downwards over a packing material while a countercurrent vapor stream resulting from the heating of the extract stream in a heater below the packing (e.g., in the same vessel or a different vessel) passes through the down coming liquid extract stream 256. The flash tank and/or packed column may operate at a pressure ranging from about 1 atm to about 80 atm, and a temperature of between about 70° C. to about 120° C., or from about 85° C. to about 100° C. In some embodiments, the separation device may comprise a distillation column. The extract stream 256 can be heated within the distillation column to dissociate the adducts into the impurities and the extracting agent within the solvent. The impurities may then be separated by distillation. The distillation column may comprise about 1 to about 100 stages, or about 1 to about 50 stages. The distillation column may operate at a pressure ranging from about 1 atm to about 80 atm. The temperature and pressure may be selected based on the equilibrium on each stage within the distillation column. In an embodiment, the temperature within the column may range from about 50° C. to about 200° C., or from about 70° C. to about 150° C.

In an embodiment, the stripping section may operate at a vacuum pressure relative to atmospheric pressure. A lower operating pressure is generally expected to reduce the temperature at which the solvent can be separated from the impurities when the adducts are dissociated. In an embodiment, the stripping section may operate at a pressure ranging from about 0.001 atm to about 1 atm and at a temperature of between about 45° C. and about 100° C. In general, the solvent and/or extracting agent may break down during the heating process in the stripping section, potentially releasing contaminants into the vapor stream. By operating at a reduced pressure, and resulting reduced temperature, the rate of solvent and/or extracting agent loss may be reduced. For example, by lowering the temperature at which the stripping occurs, the rate at which any hydrosulfite/sulfite in solution breaks down to form sulfur dioxide can be reduced or limited.

When the stripping section 304 comprises a phase separator, an overhead product stream 306 may exit the stripping section 304 and pass to a condenser 308. The overhead stream 306 may comprise the separated impurities, dissolved ethyl acetate, and potentially minor amount of water and decomposition products resulting from a breakdown of the extracting agent. Within the condenser 308, the temperature of the overhead stream 306 may be reduced to condense the impurities into a liquid, which may leave the condenser 308 as an impurities stream 312. The impurities stream 312 may exit the system for sale, for use as fuel, and/or as a feed to one or more suitable downstream processes. Any remaining gases may exit the condenser as an off-gas stream 311. The off-gas stream 311 may comprise a portion of the vaporized impurities, ethyl acetate, and any decomposition products from the solvent. The off-gas stream 311 can be vented to the atmosphere or a flare, or as shown in FIG. 5, recycled to be combined with the regenerated solvent stream 307. The regenerated solvent stream 307 may leave the stripping section 304 as a bottoms stream and pass to the contactor 314. The off-gas stream 310 may then be re-absorbed within the contactor 314. The combined regenerated solvent stream 254 may pass to a heat exchanger 316, which may comprise any of the types of heat exchangers described herein. The regenerated solvent stream 254 may be cooled within the heat exchanger 316 to a temperature suitable to allow the extracting agent within the solvent to form an adducts with any corresponding impurities in the product stream 310. The cooled, regenerated solvent stream 254 may then be combined with an optional solvent make-up stream 318 to form the solvent stream 319. The make-up stream may include the solvent comprising the extracting agent in any suitable concentration. In an embodiment, the make-up stream may serve to adjust the concentration of the extracting agent and defoamer within the solvent stream 319 to a desired level.

In some embodiments, the stripping section 304 may comprise a liquid-liquid separator. The liquid-liquid separator may operate at a pressure ranging from about 1 atm to about 80 atm. The extract stream 256 may be heated to a temperature in the range of between about 40° C. to about 120° C., or from about 45° C. to about 100 C within the separator, which may be sufficient to dissociate the adduct. The impurities may then form an organic liquid phase that is at least partially insoluble in the aqueous solvent. The resulting phases may be separated in a liquid-liquid phase separation device, such as a settling tank or settling tower. Suitable draw points may be used to provide a regenerated solvent stream 307 and an organics stream 306. The organics stream 306 may leave the system without any further separation or processing. The liquid-liquid separator may comprise a gas vent suitable for passing any off-gases back to the contactor 314. The aqueous phase may leave the liquid-liquid separator as the regenerated solvent stream 307 and pass back to the extractor through the contactor 314 and heat exchanger 316 as described above.

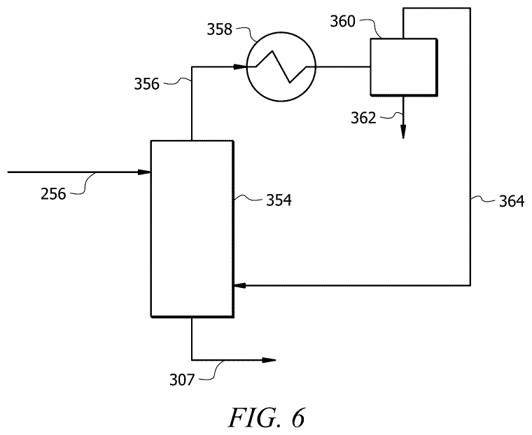

FIG. 6 illustrates an embodiment of a stripping section 354 that is similar to the stripping section 304 described with respect to FIG. 5. In the interest of clarity, similar components will not be described. The stripping section 354 may comprise any of the separation devices operating at any of the operating conditions as described above with respect to FIG. 5, and the stripping section 354 can be used with the remaining components of the extraction process 350 illustrated in FIG. 5. The stripping section 354 differs from the stripping section 304 of FIG. 5 in that a purge gas stream 364 can be introduced into the stripping section 354, which can aid in the extraction of the impurities from the solvent solution. In an embodiment, the purge gas can comprise a gas having a boiling point significantly below that of the impurities, and the purge gas may have a low solubility in the solvent and/or impurities to aid in separation from the solvent and impurities. The purge gas can include, but is not limited to, nitrogen, oxygen, argon, helium, hydrogen, carbon dioxide, and any combination thereof (e.g., air). The use of the purge gas may aid in removing the impurities from the extract stream 256 while maintaining a relatively low stripping temperature. The use of the low temperature may aid in limiting or reducing the breakdown of the solvent and/or extracting agent, which can potentially resulting the generation of pollutants.

The extract stream 256 can be introduced into the stripping section 354 and can include a solvent fluid (e.g., which can comprise the extracting agent and defoamer), the adducts formed by the reaction between the extracting agent and the impurities, any excess extracting agent, and potentially some amount of dissolved ethyl acetate. As the liquid extract stream 256 flows through the stripping section, a purge gas stream 364 can be introduced into the lower portion of the stripping section 354. The ratio of the purge gas volumetric flow rate to the extract stream volumetric flowrate can range from about 1,000:1 to about 1:100, and the ratio may be based on the amount of impurities present in the extract stream, the conditions within the stripping section 354, and/or the solubility of the impurities in the purge gas. The extract stream 256 can be heated to a temperature in the range of about 45° C. to about 100° C. before and/or within the stripping section 354, resulting in at least a partial dissociation of the adducts. Vapor-liquid stripping can then take place between the impurities released from the adducts and the purge gas. Since the impurities would generally have a lower boiling point than the solvent, the impurities could expect to be stripped into the purge gas stream.

The stripping section 354 can have an overhead product stream 356 that can pass to a cooler 358 and into a separator 360. The overhead stream 356 may comprise the purge gas, impurities stripped by the purge gas, and potentially minor amount of solvent, ethyl acetate, and decomposition products resulting from a breakdown of the extracting agent. Within the cooler 358, the temperature of the overhead stream 356 may be reduced to condense the impurities into a liquid, which may then pass to the separator 360 and leave as an impurities stream 362. The impurities stream 362 may exit the system for sale, for use as fuel, and/or as a feed to one or more suitable downstream processes. The purge gas may not condense and may pass out of the separator 360 as the purge gas stream 364 for recycle to the stripping section 354. The purge gas stream 364 may comprise a minor amount of the impurities, the solvent, and the decomposition products, that can reach an equilibrium in the process due to the recycling of the purge gas stream 364. The regenerated solvent stream 307 can be processed and/or recycled as described with respect to the regenerated solvent stream in FIG. 5.

The extraction section may comprise a number of flow configurations. The extraction may be carried out in a co-current, counter-current, and/or cross-current flow scheme, as schematically illustrated in FIGS. 7, 8, and 9, respectively. An embodiment of a co-current extraction scheme is illustrated in FIG. 7. In this embodiment, the extraction section may comprise a plurality of stages 402, 404, 406. For each stage 402, 404, 406, the product stream 310 and the solvent stream 319 may enter the stage and flow together through the stage. The solvent and product may be separated within the stage, for example using a settler/phase separator. Each phase may then pass to the next stage as separate phases. Alternatively, the combined fluids may pass to the next stage as a single stream. In general, the co-current flow of solvent and product may allow the two streams to reach an equilibrium, and therefore, the co-current flow may only provide an extraction equivalent to a single theoretical stage. However, additional stages may be useful in practice to provide additional residence/contact time to approach the equilibrium concentrations and mass transfer of the impurities from the ethyl acetate product into the solvent phase. The solvent phase may be separated from the product phase within the last stage or within a separator downstream of the last stage to produce the extract stream 256 and the purified product stream 216. While illustrated as separate stages 402, 404, 406, the stages may be present in a single physical vessel. Further, while three stages 402, 404, 406 are shown in FIG. 7, less than three stage or more than three stages may be used. In an embodiment, the number of stages may be between about 1 and about 50 stages.

An embodiment of a counter-current extraction scheme is illustrated in FIG. 8. In this embodiment, the extraction section may comprise a plurality of stages 408, 410, 412. The product stream 310 may enter at a first of the stages and the solvent stream 319 may enter the last stage and flow counter-currently through each stage. The phases may be arranged to allow the phases to flow counter-currently based on density differences, with the organic product phase flow upwards and the aqueous solvent phase flow downwards. The countercurrent flow of the product and solvent may result in the most efficient extraction and mass transfer of the three flow schemes. The solvent phase may be separated from the product phase within the first stage 408 to allow the extract stream 256 to leave the extraction section 302. Similarly, the product phase may be separated from the solvent phase within the last stage 412 to allow the purified product stream 216 to leave the extraction section 302. While illustrated as separate stages 408, 410, 412, the stages may be present in a single physical vessel. Further, while three stages 408, 410, 412 are shown in FIG. 8, less than three stage or more than three stages may be used. In an embodiment, the number of stages may be between about 1 and about 50 stages.

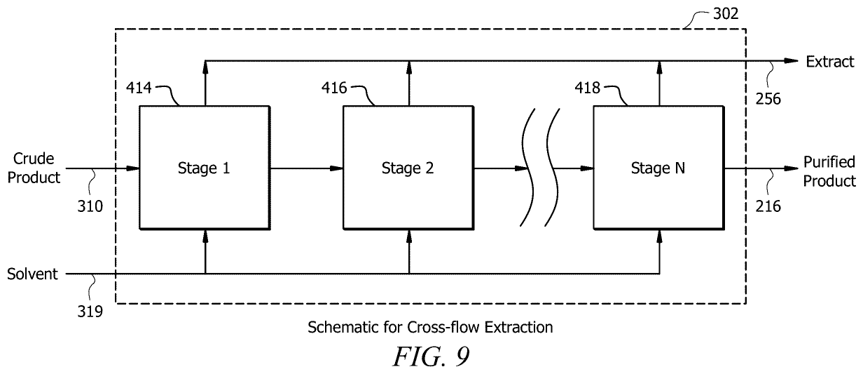

An embodiment of a cross-current extraction scheme is illustrated in FIG. 9. In this embodiment, the extraction section may comprise a plurality of stages 414, 416, 418. The product stream 310 may enter at a first of the stages and progress through each stage 414, 416, 418. The solvent stream 319 can be divided into portions, which may have the same or different flow rate. The portions may pass to each stage 414, 416, 418, respectively, to contact the product within each stage 414, 416, 418. Within each stage 414, 416, 418, the solvent phase may be separated from the product phase, and the solvent phase portions may be recombined to form extract stream 256. The solvent phase may pass out of the last stage 418 as the purified product stream 216. The use of the cross-current flow configuration may allow the concentration of the impurities in the ethyl acetate product stream to be decreased in each stage. In an embodiment, the cross-current extraction scheme may comprise separate vessels (e.g., contactors, settlers, etc.) for each stage. In some embodiments, the stages may be present in a single physical vessel with internal contactors and separators. Further, while three stages 408, 410, 412 are shown in FIG. 9, less than three stage or more than three stages may be used. In an embodiment, the number of stages may be between about 1 and about 50 stages.

While described separately, the extraction section 302 may comprise any combination of the extraction schemes illustrated in FIGS. 7, 8, and 9. For example, each stage of the cross-current flow scheme may comprise a co-current or counter-current individual flow scheme. Various other combinations may also be possible.

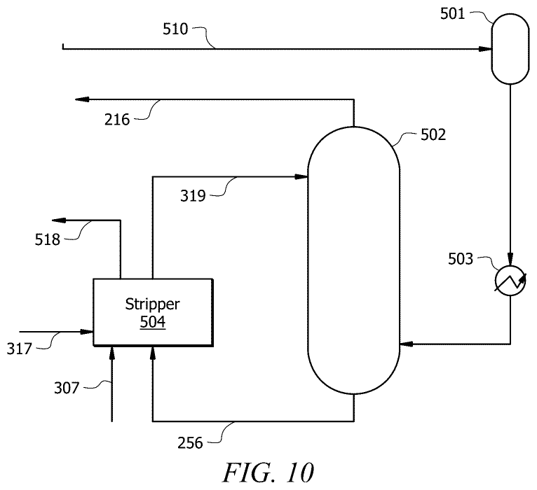

FIG. 10 illustrates another embodiment of an extraction process that can be used to extract various byproducts such as 2-butanone (which is also referred to as methyl ethyl ketone (MEK)) from a crude ethyl acetate stream. In this embodiment, a product stream 510 is fed to an extraction section 502. The product stream may generally correspond to the crude product stream 210 or any other stream comprising ethyl acetate and one or more impurities, including any of those described herein. The product stream 510 may predominantly comprise ethyl acetate, though various byproducts including any of those described herein such as ketones and/or aldehydes such as 2-butanone may also be present.

The product stream 510 can pass to a surge vessel 501 to provide temporary storage for fluctuating process flows. The product stream can then pass to a cooler 503 to adjust the temperature of the product stream 510 to a desired temperature for the extraction process. In some aspects, the product stream 510 temperature can be adjusted to a temperature in the range of about a temperature of between about 0° C. to about 70° C., from about 10° C. to about 60° C., or from about 20° C. to about 50° C. before passing to the extraction column 502.

The extraction column 502 is illustrated in FIG. 10 as a countercurrent liquid-liquid column (e.g., a Sheibel column, etc.), though any of the liquid-liquid contactors described herein can be used. Within the extraction column 502, the product stream 510 can be contacted with a solvent, which can comprise any of the components described herein. The extraction may be carried out in a co-current, counter-current, and/or cross-current flow scheme. In an embodiment as shown in FIG. 10, the extraction column 502 can comprises a counter-current flow arrangement between the solvent and product streams.

A contact device within the extraction section may comprise a number of stages configured to provide the degree of impurity removal from the product stream 510 and/or a desired purity of the purified ethyl acetate product stream. In an embodiment, the extraction column 502 can contain an equivalent to between about 1 to about 100 theoretical stages. The extraction column 502 can operate at any suitable pressure. In an embodiment, the extraction column 502 may operate at a pressure ranging from about 1 atm to about 80 atm, and a temperature of between about 0° C. to about 70° C., from about 10° C. to about 60° C., or from about 20° C. to about 50° C.

Within the extraction column 502, at least a portion of the impurities such as the 2-butanone may be transferred to the solvent. In an embodiment, the extracting agent may react with a portion of the impurities in the product stream 510 to form an adduct and improve the solubility of the impurity in the solvent phase. Foam resulting from one or more components of the resulting mixture can be controlled, reduced, or prevented due to the presence of the defoamer in the solvent. Once the product stream 510 and the solvent stream 319 have been contacted and separated within the extraction column 502, a purified product stream 216 and an extract stream 256 may exit the extraction column 502. The purified product stream 216 may have an increased purity of ethyl acetate relative to the entering product stream 510. As described above, the purified product stream 216 may pass to a downstream separation section to further purify the purified product stream. In an embodiment, the purified product stream 216 and/or a dried, purified product stream may comprise ethyl acetate having a purity of greater than about 90%, greater than about 95%, greater than about 96%, greater than about 97%, greater than about 98%, greater than about 99%, or greater than about 99.5% ethyl acetate by weight.

The extract stream 256 may comprise the solvent and the impurities removed from the product stream 510 such as at least a portion of the 2-butanone. The extract stream 256 may pass to the stripping section 504. While the defoamer can be added at any point in the circulation of the solvent between the extraction and stripping sections, the defoamer in stream 317 can be added in some embodiments in one or more streams around or within the stripping section 504. Additional solvent or components of the solvent can be added in makeup solvent stream 318. In an embodiment, the extract stream 256 may comprise a solvent fluid, the adducts formed by the reaction between the extracting agent and the impurities, any excess extracting agent, the defoamer, and potentially some amount of dissolved ethyl acetate. The stripping section 504 may comprise one or more separation devices configured to separate the impurities from the solvent and any extracting agent including any of those described herein. In an embodiment, the stripping section may operate at a vacuum pressure relative to atmospheric pressure. A lower operating pressure is generally expected to reduce the temperature at which the solvent can be separated from the impurities when the adducts are dissociated. In an embodiment, the stripping section may operate at a pressure ranging from about 0.001 atm to about 1 atm and at a temperature of between about 45° C. and about 100° C.

The stripping section can produce a lean solvent stream 319 that can pass back to the extraction column 502. An impurities stream 518 can comprise the portion of the impurities removed from the crude product stream 510. The impurities stream 518 can pass to a storage tank for the impurities and/or other impurities removed in the system.

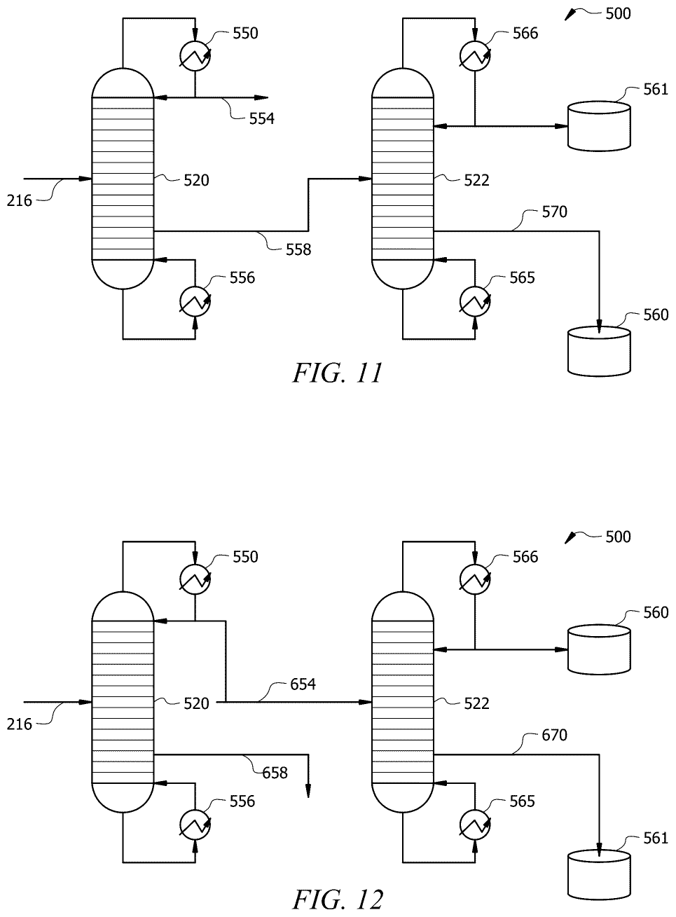

The purified ethyl acetate stream from any of the byproduct removal sections described herein can then pass to a product purification section 500. An embodiment of a product purification section is shown in FIG. 11. As shown, a product separation section 500 may comprise a plurality of separation units configured to further purify the ethyl acetate and remove impurities including light impurities and heavy impurities. As shown, the purified product stream may enter separator 520. The purified product stream 216 may be at a desired temperature based on the operating conditions of the byproduct removal section 400, or alternatively, the temperature and/or pressure can be adjusted using one or more heat exchangers, valves, pumps, or the like upstream of the product purification section 500. The separator 520 can be a phase separator, which is a vessel that separates an inlet stream into a substantially vapor stream and a substantially liquid stream, such as a knock-out drum, flash drum, distillation column, reboiler, condenser, or other heat exchanger. Such vessels also may have some internal baffles, trays, packings, temperature control elements, and/or pressure control elements. The separator 520 also may be any other type of separator, such as a membrane separator. In a specific embodiment, the separator 520 comprises a distillation column. The separator 520 may generally operate at a pressure of between about 1 atm to about 200 atm. In an embodiment, the separator 520 operates at a pressure below the pressure of separator 522 as described below. The temperature profile in the column may be dictated by the mixture boiling point along the height of the column. In some aspects, the temperature within the column can range between about 30° C. to about 15° C., or alternatively between about 5° C. and about 10° C. The separator 520 may separate the purified product stream 216 into an overhead stream and a bottoms stream. The overhead stream may pass through a heat exchanger 550 to cool and condense the overhead stream. A portion of the overhead stream can be returned to the separator 520 as reflux while the remaining portion in stream 554 can pass to a byproduct storage tank (e.g., storage tank 560, etc.). The overhead stream may predominantly comprise ethyl acetate with minor amounts of acetaldehyde, ethanol, water, and other light components such as acetone, diethyl ether, and/or other acetates. In some aspects, the products in stream 554 can be between about 1 to about 10 wt. %, or alternatively, between about 3 to about 8 wt. % of the entering purified product stream 216. An optional reboiler 556 can be in fluid communication with a lower portion of the separator 520 to vaporize a portion of the fluid within the column to produce a rising vapor stream within the separator 520. The bottoms stream 558 can pass out of the separator 520 to a heavies removal separator 522. In some aspects, the relative amount of light byproducts being removed may not require the presence of the reboiler, and the bottoms stream 558 can pass directly to the heavies removal separator 520 without the use of a reboiler with separator 520. The bottoms stream 558 may predominantly comprise ethyl acetate in addition to minor amounts of butanol, higher alcohols, and/or additional heavier reaction products. In some aspects, the heavier byproducts such as butanol, ethyl butyrate, N-butyl acetate, and/or heavier acetates may be present with the ethyl acetate in the bottoms stream in a total amount of between about 1 to about 8 wt. % or between about 2 to about 4 wt. %.

Separator 522 may comprise any of the separators described herein, and, in an embodiment, separator 522 comprises a distillation column. Separator 522 may separate the bottoms stream 558 into an overhead stream comprising ethyl acetate product and a bottoms stream 570 comprising the higher boiling components including butanol, higher alcohols, and other reaction byproducts. In some aspects, the temperature within the separator 522 can range between about 35° C. to about 150° C., or alternatively between about 40° C. and about 120° C., and the pressure can range from about 1 atm to about 200 atm, or between about 1 atm and about 50 atm. The overhead stream can pass through a condenser 566 to at least partially condense the overhead stream. A first portion of the condensed overhead stream can pass back to the separator 522 as a reflux stream, and the remaining ethyl acetate product stream can pass to a storage battery 561 where the product can be stored prior to sales. A bottoms stream can pass to a reboiler 565 to produce a vapor phase reflux o the separator 522. The bottoms stream 570 can pass to the byproduct storage tank 560 where the heavies produced in the process may exit the system for use as fuel, as a final product, and/or as a feed to one or more suitable downstream processes.

In some embodiments, the heavies byproduct stream can comprise a minor amount of ethyl acetate such as between about 1 to 10 wt. % with the remainder being butanol, ethyl butyrate, and higher boiler acetates (e.g., butyl acetate, isopropyl acetate, etc.). The relative mass flow of the heavies product stream 570 can be between about 1 to 5 wt. %, or between about 2 to 4 wt. % of the mass flowrate of the bottoms stream 558 entering the separator 522. In an embodiment, the ethyl acetate product stream may comprise greater than about 90%, greater than about 95%, greater than about 96%, greater than about 97%, greater than about 98%, greater than about 99%, greater than about 99.5%, or greater than about 99.8% ethyl acetate by weight. One or more additional separation processes can optionally be used to further purify the ethyl acetate product to greater than 99.9 wt. % or greater than 99.995 wt. %. In the process as shown in FIG. 11, greater than about 96 wt. %, greater than about 97 wt. %, greater than about 98 wt. %, or greater than about 98.5 wt. % of the ethyl acetate in the purified product stream 216 can be recovered as the ethyl acetate produce in the product storage 561.

The system described with respect to FIG. 12 can also be operated with the first separator 520 operated to remove the heavier boiling components and the second separator 522 being operated to remove the light boiling components. Referring to the embodiment of the product purification section as shown in FIG. 12, a product separation section 500 may comprise a plurality of separation units configured to further purify the ethyl acetate and remove impurities including light impurities and heavy impurities. In use, the purified product stream 216 (or alternatively stream 516) may enter separator 520. The purified product stream 216 may be at a desired temperature based on the operating conditions of the byproduct removal section 400, or alternatively, the temperature and/or pressure can be adjusted using one or more heat exchangers, valves, pumps, or the like upstream of the product purification section 500. The separator 520 can be a phase separator, and in a specific embodiment, the separator 520 comprises a distillation column. The separator 520 may generally operate at a pressure of between about 1 atm to about 200 atm. The temperature profile in the separator 520 may be dictated by the mixture boiling point along the height of the column. In some aspects, the temperature within the column can be between about 30° C. to about 150° C., or alternatively between about 40° C. and about 80° C. The separator 520 may separate the purified product stream 216 into an overhead stream and a bottoms stream. The overhead stream may pass through a heat exchanger 550 to cool and condense the overhead stream. A portion of the overhead stream can be returned to the separator 520 as reflux while the remaining portion in stream 654 can pass to the second separator 522. The overhead stream may predominantly comprise ethyl acetate with minor amounts of acetaldehyde, ethanol, water, and other light components such as acetone, diethyl ether, and/or other acetates. In some aspects, the products in stream 654 can have a mass flowrate of between about 1 to about 10 wt. %, or alternatively, between about 3 to about 8 wt. % of the mass flowrate of the purified product stream 216.

A reboiler 556 can be in fluid communication with a lower portion of the separator 520 to vaporize a portion of the fluid within the column to produce a rising vapor stream within the separator 520. The bottoms stream 658 can pass out of the separator 520 to the byproduct storage tank 560. The bottoms stream 658 may predominantly comprise heavy boiling components in addition to minor amounts of ethyl acetate. The heavy boiling components can include, but are not limited to, butanol, higher alcohols, and/or additional heavier reaction products. In some aspects, the heavier byproducts such as butanol, ethyl butyrate, N-butyl acetate, and/or heavier acetates may be present in the bottoms stream in a total amount of between about 80 to about 96 wt. % or between about 90 to about 97 wt. %.

Separator 522 can receive the overhead stream 654 from the first separator 520. Separator 522 may comprise any of the separators described herein, and, in an embodiment, separator 522 comprises a distillation column. Separator 522 may separate the overhead stream 654 into a bottoms stream 670 comprising ethyl acetate product and an overhead stream comprising the higher boiling components including acetaldehyde, ethanol, water, and lighter components such as acetone and/or diethyl ether. In some aspects, the temperature within the separator 522 can range between about 35° C. to about 150° C., or alternatively between about 40° C. and about 100° C., and the pressure can range from about 1 atm to about 200 atm, or between about 1 atm and about 50 atm. The overhead stream can pass through a condenser 566 to at least partially condense the overhead stream. A first portion of the condensed overhead stream can pass back to the separator 522 as a reflux stream, and the remaining lights byproduct stream can pass to a byproduct storage 560. A bottoms stream 670 can optionally pass to a reboiler 565 to produce a vapor phase reflux o the separator 522. In some aspects, the relative amount of light byproducts being removed in separator 522 may not require the presence of the reboiler, and the bottoms stream 670 can pass directly to the outlet without the use of a reboiler with separator 522. The bottoms stream 670 can pass to the product storage tank 561.

In some embodiments, the lights byproduct stream can predominantly ethyl acetate with the remainder being lighter boiling components such as acetaldehyde, water, and the like. The light byproducts can comprise between about 5 to about 15 wt. % of the overhead stream. The relative mass flow of the light byproduct stream can be between about 1 to 8 wt. %, or between about 3 to 6 wt. % of the mass flowrate of the overhead stream 654 entering the separator 522. In an embodiment, the ethyl acetate product stream 670 may comprise greater than about 90%, greater than about 95%, greater than about 96%, greater than about 97%, or greater than about 98%, greater than about 99%, or greater than about 99.5% ethyl acetate by weight. One or more additional separation processes can optionally be used to further purify the ethyl acetate product to greater than 99.9 wt. % or greater than 99.98 wt. %. In the process as shown in FIG. 12, greater than about 96 wt. %, greater than about 97 wt. %, greater than about 98 wt. %, greater than about 98.5 wt. %, or greater than about 98.8 wt. % of the ethyl acetate in the purified product stream 216 can be recovered as the ethyl acetate produce in the product storage 561.

While the embodiments as shown in FIGS. 11 and 12 are described as receiving the purified product stream, the systems and methods described with respect to FIGS. 11 and 12 can also be used with the crude ethyl acetate stream from the separator 201 directly without the use of the extraction system. The byproducts removed in the extraction system would then be removed with the other heavy byproducts and stored in the byproduct storage 560.

### Dehydrogenation and Dimerization Catalysts

Suitable dehydrogenation and dimerization catalysts are capable of converting at least a portion of the alcohol (e.g., ethanol) in a feed stream to a higher valued product such as ethyl acetate, and the catalysts can be present in any one of the reactors in the reactor section 200. Any catalyst capable of carrying out a dehydrogenation and dimerization reaction may be used alone or in combination with additional catalytic materials in the reactors. In an embodiment, suitable dehydrogenation and dimerization catalysts can generally comprise metals and/or oxides of copper, barium, ruthenium, rhodium, platinum, palladium, rhenium, silver, cadmium, zinc, zirconium, gold, thallium, magnesium, manganese, aluminum, chromium, nickel, iron, molybdenum, sodium, strontium, tin, and mixtures thereof. In many cases, the catalyst material will be provided on a support material. The catalyst can be treated with a carbonate (e.g., sodium carbonate), reduced with hydrogen, and/or other suitable treatments prior to use.

In certain embodiments, the dehydrogenation and dimerization catalyst may include a catalyst support. The catalyst support stabilizes and supports the catalyst. The type of catalyst support used depends on the chosen catalyst and the reaction conditions. Suitable supports may include, but are not limited to, carbon, silica, silica-alumina, alumina, zirconia, titania, ceria, vanadia, nitride, boron nitride, heteropolyacids, hydroxyapatite, zinc oxide, chromia, zeolites, carbon nanotubes, carbon fullerenes, and any combination thereof.

The dehydrogenation and dimerization catalyst can be employed in any of the conventional types or structures known to the art. It may be employed in the form of extrudates, pills, pellets, granules, broken fragments, or various special shapes. In an embodiment, consideration of the use of the catalyst in the reactive distillation system and/or as a mass transfer surface within the distillation column may be taken into account when determining a suitable shape. For example, the catalyst may have a shape similar to structured packing material or suitable for insertion in a structured packing. When the hydrogenation catalyst is used with one or more side reactors, the catalyst may be disposed within a reaction zone, and the feed may be passed therethrough in the liquid, vapor, or mixed phase, and in either upward or downward, or inward or outward flow.

The dehydrogenation and dimerization catalyst may typically have a range of metal loadings. In an embodiment, the catalyst may have a copper oxide weight loading (i.e., weight percentage) of between about 0.5% and about 90%, between about 10% and about 70%, between about 20% and about 65%, between about 30% and about 60%, or about 40% and about 50%. In an embodiment, the catalyst may have a zinc oxide weight loading of between about 20% and about 60%, between about 30% and about 50%, or between about 40% and about 50%. In an embodiment, the catalyst may have a chromium oxide weight loading of between about 20% and about 60%, or between about 30% and about 50%.

In an embodiment, the catalyst may comprise CuO/ZnO/Al₂O₃. In this embodiment, the catalyst may have a copper oxide weight loading of between about 0.5% and about 90%, between about 10% and about 70%, between about 20% and about 65%, between about 30% and about 60%, or about 40% and about 50%, and the zinc oxide and alumina may comprise the balance of the weight. In an embodiment, the catalyst may comprise CuO/ZnO/ZrO₂/Al₂O₃, and the catalyst may have a copper oxide weight loading of between about 40% to about 90%, with the remainder of the components forming the balance of the catalyst weight. In some aspects, the CuO/ZnO/ZrO₂/Al₂O₃ catalyst may also comprise a conductive carbon such as graphite, and the overall composition may have between about 50-70 wt. % copper oxide, 15-25 wt. % zinc oxide, 5-15 wt. % aluminum oxide, 3-13 wt. % zirconium dioxide, and 1-6 wt. % conductive carbon (e.g., graphite). In an embodiment, the catalyst may comprise CuO/ZnO/ZrO₂/Cr₂O₃, and the catalyst may have a copper oxide weight loading of between about 20% to about 90% and a chromium oxide weight loading between about 30% and about 50%, with the remainder of the components forming the balance of the catalyst weight. In an embodiment, the catalyst may comprise CuO/ZrO₂/Al₂O₃. In an embodiment, the catalyst comprises an alkaline earth metal and/or alkaline earth metal oxide and copper and/or copper oxide on a support. In this embodiment, the support may comprise silica.

Any of the materials useful as dehydrogenation and dimerization catalysts, may be synthesized using a variety of methods. In an embodiment, the dehydrogenation and dimerization catalyst may be prepared via wet impregnation of a catalyst support. Using the wet-impregnation technique, a metal nitrate dissolved in a suitable solvent may be used to prepare the catalyst, however any soluble compound would be suitable. A sufficient amount of solvent should be used to fully dissolve the metal nitrate and appropriately wet the support. In one embodiment, copper nitrate and ethanol and/or water may be mixed in an amount sufficient such that the copper nitrate dissolves. Additional metal nitrates may also be added to provide a catalyst with additional components. The solute may then be combined with a suitable support material of appropriate particle size. The mixture may then be refluxed at a temperature of approximately 100° C. for approximately several hours (e.g., three to five hours) and then allowed to dry at a temperature of about 110° C. The dried material may then be heated to 200° C. to remove the NOx component, and then the materials may be calcined at about 450° C. to about 550° C. at a heating rate of about one to ten ° C./min. The amount of metal nitrate used in the wet-impregnation technique can be adjusted to achieve a desired final metal weight loading of the catalyst support.

When multiple components are used to provide a catalyst disposed on a support, each component can be added via the wet-impregnation technique. The appropriate salts can be dissolved and impregnated on a support in a co-impregnation process or a sequential process. In a co-impregnation process, measured amount of the appropriate plurality of metal salts may be dissolved in a suitable solvent and used to wet the desired catalyst support. The impregnated support can then be dried and calcined to provide a final catalyst with a desired weight loading. In the sequential impregnation process, one or more measured amounts of salts may be dissolved in a suitable solvent and used to wet the desired catalyst support. The impregnated support can then be dried and calcined. The resulting material can then be wetted with one or more additional salts that are dissolved in a suitable solvent. The resulting material can then be dried and calcined again. This process may be repeated to provide a final catalyst material with a desired loading of each component. In an embodiment, a single metal may be added with each cycle. The order in which the metals are added in the sequential process can be varied. Various metal weight loadings may be achieved through the wet-impregnation technique. In an embodiment, the wet-impregnation technique may be used to provide a catalyst having a copper weight loading ranging from about 0.5% and about 50%, with one or more additional components having a weight loading between about 0.1% and about 10%.

The dehydrogenation and dimerization catalysts may also be prepared via a co-precipitation technique. In this technique, a measured amount of one or more appropriate metal nitrates (or other appropriate metal salts) are dissolved in de-ionized water. The total metal concentration can vary and may generally be between about 1 M and about 3 M. The metal-nitrate solution may then be precipitated through the drop-wise addition of the solution to a stirred, equal volume of a sodium hydroxide solution at room temperature. The sodium hydroxide solution may generally have a concentration of about 4M, though other concentrations may also be used as would be known to one of skill in the art with the benefit of this disclosure. After addition of the metal nitrate solution, the resulting suspension can be filtered and washed with de-ionized water. The filtered solids can be dried overnight, for example, at a temperature of about 110° C. The resulting mixed metal oxide can then be processed to a desired particle size. For example, the resulting mixed metal oxide can be pressed to a desired form, ground, and then sieved to recover a catalyst material with a particle size in a desired range. Catalysts prepared using the co-precipitation technique may have higher metal loadings than the catalysts prepared using the wet-impregnation technique.

The catalyst prepared via the co-precipitation technique may be used in the prepared form and/or a catalyst binder can be added to impart additional mechanical strength. In an embodiment, the prepared catalyst may be ground to a fine powder and then stirred into a colloidal suspension (e.g., a colloidal suspension of silica and/or alumina) in an aqueous solution. The resulting suspension may be stirred while being heated and allowed to evaporate to dryness. The heating may take place at about 80° C. to about 130° C. The resulting solid can then be processed to a desired particle size. For example, the resulting solid can be pressed to a desired form, ground, and then sieved to recover a catalyst material with a particle size in a desired range. Alternatively, the colloidal suspension may be added to the 4M sodium hydroxide precipitation solution prior to addition of the metal nitrate solution in the co-precipitation technique. Various metal weight loadings may be achieved through the co-precipitation technique. In an embodiment, the co-precipitation technique may be used to provide a catalyst having a copper weight loading ranging from about 2% and about 80%, with one or more additional components having a weight loading between about 2% and about 40%.

The resulting catalyst from either the wet-impregnation technique and/or the co-precipitation technique may be further treated prior to use in the reactive distillation system disclosed herein. In an embodiment, the catalyst may be treated with a sodium carbonate solution for a period of time to improve the selectivity of the catalyst. In this process, the catalyst may be soaked in an aqueous solution of sodium carbonate for a period of time ranging from about 1 hour to about 48 hours, or alternatively about 2 hours to about 24 hours. In an embodiment, the sodium carbonate solution may have a concentration of about 0.2 M. The catalyst may then be filtered and allowed to dry at about room temperature. In an embodiment, the sodium carbonate may comprise from about 0.2 to about 3.0 weight percent of the catalyst after being contacted with the sodium carbonate solution.

In another treatment process, the catalyst may be reduced with hydrogen prior to use. In this embodiment, the catalyst may be heated and contacted with hydrogen, which may be flowing over the catalyst, for a period of time sufficient to reduce the catalyst to a desired degree. In an embodiment, the catalyst may be contacted with hydrogen at a temperature of about 160° C. to about 240° C. The hydrogen treatment may be conducted in combination with the sodium carbonate treatment, and may be performed prior to and/or after the sodium carbonate treatment.

Without intending to be limited by theory, it is believed that the production of hydrogen during the dehydrogenation and dimerization reaction within the process may result in contact between the dehydrogenation and dimerization catalyst and a hydrogen stream sufficient to at least partially reduce the catalyst. Thus, the process described herein may have the potential for the in-situ reduction of the catalyst during use. This may result in an initial break-in period in which the catalyst conversion and selectivity may change before reaching a steady state conversion and selectivity. This in-situ reduction may be taken into account when considering the degree to which a catalyst should be pre-reduced with hydrogen.

Having described the systems and methods, various embodiments may include, but are not limited to:

In a first aspect, a method of purifying an ethyl acetate stream comprises: contacting an inlet stream with a solvent, wherein the inlet stream comprises ethyl acetate and an impurity compound, and wherein the solvent comprises an extracting agent and a defoamer; transferring at least a portion of the impurity compound from the inlet stream into the solvent to form an extract and a purified product, wherein the extract comprises the solvent and the portion of the impurity compound transferred from the inlet stream; separating the extract from the purified product; separating the portion of the impurity compound from the extract; preventing the formation of foam while separating the portion of the impurity compound from the extract using the defoamer; forming an impurities stream and a regenerated solvent; and recycling at least a portion of the regenerated solvent to contact the inlet stream.

A second aspect can include the method of the first aspect, wherein the solvent comprises an aqueous fluid.

A third aspect can include the method of the first or second aspect, wherein the extracting agent is configured to increase the solubility of the impurity compound in the solvent.

A fourth aspect can include the method of the third aspect, wherein the extracting agent comprises sodium hydrogen sulfite.

A fifth aspect can include the method of the third aspect, wherein the extracting agent comprises a compound selected from the group consisting of: hydrosulfite ion, sulfite ion, hydrazine, hydroxylamine, semicarbazide, phenylhydrazine, phenylhydroxylamine, salts thereof, aqueous solutions thereof, and any combination thereof.

A sixth aspect can include the method of any one of the third to fifth aspects, further comprising reacting the extracting agent and the impurity compound to form an adduct in response to contacting the inlet stream with the solvent.

A seventh aspect can include the method of the sixth aspect, wherein separating the portion of the impurity compound from the extract comprises dissociating the adduct.

An eighth aspect can include the method of any one of the first to seventh aspects, wherein the impurity compound comprises at least one of a ketone, an aldehyde, or any combination thereof.

A ninth aspect can include the method of any one of the first to eighth aspects, wherein contacting the inlet stream with the solvent comprises at least one of contacting the inlet stream with the solvent in a counter-current flow, or contacting the inlet stream with the solvent in a cross-current flow.

A tenth aspect can include the method of any one of the first to ninth aspects, further comprising: feeding a feed stream comprising ethanol to one or more reactors; contacting the ethanol with a catalyst in the one or more reactors; dehydrogenating ethanol over the catalyst; producing ethyl acetate and the impurity compound during the dehydrogenation to produce an effluent stream comprising the ethyl acetate and the impurity compound; removing hydrogen from the effluent stream; and providing the effluent stream as the inlet stream.

An eleventh aspect can include the method of any one of the first to tenth aspects, wherein the defoamer comprises a primary alcohol and an alcohol alkoxylate.

A twelfth aspect can include the method of the eleventh aspect, wherein the primary alcohol comprises one or more C₁₄ to C₃₂ primary linear alcohols.

A thirteenth aspect can include the method of the eleventh or twelfth aspect, wherein the alcohol alkoxylate comprises a hydrophobic group and a hydrophilic group.

A fourteenth aspect can include the method of the eleventh aspect, where the defoamer comprises a silicone based defoamer.

In a fifteenth aspect, a system for producing high purity ethyl acetate from ethanol comprises: a heater, wherein the heater is configured to heat an inlet ethanol stream; at least one reactor comprising a dehydrogenation catalyst, an ethanol inlet configured to pass ethanol in the inlet ethanol stream from the heater over the dehydrogenation catalyst to produce a product stream comprising ethyl acetate and impurities; an extraction unit configured to receive the product stream from the at least one reactor, contact a liquid solvent feed stream with the product stream, provide an extract stream comprising a portion of the impurities in the product stream, and provide a purified product stream, wherein the liquid solvent feed stream comprises an extracting agent and a defoamer; and a stripping unit configured to receive the extract stream from the extraction unit, separate the impurities from the extract stream, provide an outlet impurities stream, and provide a regenerated solvent stream back to the extraction unit as at least a portion of the liquid solvent feed stream.

A sixteenth aspect can include the system of the fifteenth aspect, further comprising a drying unit, wherein the drying unit is configured to receive the purified product stream from the extraction unit, remove at least a portion of any water in the purified product stream, and provide a dried purified product stream.

A seventeenth aspect can include the system of the fifteenth or sixteenth aspect, wherein the extraction unit comprises a liquid-liquid contact vessel.

An eighteenth aspect can include the system of any one of the fifteenth to seventeenth aspects, wherein the stripping unit comprises a distillation column.

In a nineteenth aspect, a method of producing ethyl acetate comprises feeding a feed stream comprising ethanol to one or more reactors; contacting the ethanol with a catalyst in the one or more reactors; producing ethyl acetate and one or more byproducts during the dehydrogenation to produce an effluent stream comprising the ethyl acetate and the one or more byproducts; separating a portion of the one or more byproducts to produce a purified product stream, wherein the solvent comprises an extracting agent and a defoamer; and separating at least a portion of the byproducts from the purified product stream using one or more distillation columns.

A twentieth aspect can include the method of the nineteenth aspect, wherein separating the portion of the one or more byproducts to produce the purified product stream comprises using liquid-liquid extraction in the presence of a solvent to produce the purified product stream.

A twenty first aspect can include the method of the nineteenth or twentieth aspect, wherein the byproducts comprise a first portion having boiling points above the boiling point of ethyl acetate and a second portion having boiling points below the boiling point of ethyl acetate, and wherein separating at least the portion of the byproducts from the purified product stream comprises: separating the first portion of the byproducts in a first distillation column to produce a first overhead stream comprising a majority of the first portion of the byproducts, and a first bottoms stream comprising a majority of the ethyl acetate and the second portion of the byproducts; and separating the second portion of the byproducts from the first bottoms stream in a second distillation column to produce a second overhead stream comprising a majority of the ethyl acetate and a second bottoms stream comprising a majority of the second portion of the byproducts.

A twenty second aspect can include the method of the twenty first aspect, wherein the second overhead stream comprises at least 95 wt. % of a mass flowrate of the ethyl acetate in the purified product stream.

A twenty third aspect can include the method of any one of the nineteenth to twenty second aspects, wherein the byproducts comprise a first portion having boiling points above the boiling point of ethyl acetate and a second portion having boiling points below the boiling point of ethyl acetate, and wherein separating at least the portion of the byproducts from the purified product stream comprises: separating the second portion of the byproducts in a first distillation column to produce a first overhead stream comprising a majority of the first portion of the byproducts and the ethyl acetate, and a first bottoms stream comprising a majority of the second portion of the byproducts; and separating the first portion of the byproducts from the first overhead stream in a second distillation column to produce a second overhead stream comprising a majority of the first portion of the byproducts and a second bottoms stream comprising a majority of the ethyl acetate.

A twenty fourth aspect can include the method of the twenty third aspect, wherein the second bottoms stream comprises at least 95 wt. % of a mass flowrate of the ethyl acetate in the purified product stream.

In the preceding discussion and in the claims, the terms “including” and “comprising” are used in an open-ended fashion, and thus should be interpreted to mean “including, but not limited to . . . ”. At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention.

What is claimed is:

1. A method of purifying an ethyl acetate stream comprising:
contacting an inlet stream with a solvent, wherein the inlet stream comprises ethyl acetate and an impurity compound, and wherein the solvent comprises an extracting agent and a defoamer;
transferring at least a portion of the impurity compound from the inlet stream into the solvent to form an extract and a purified product, wherein the extract comprises the solvent and the portion of the impurity compound transferred from the inlet stream;
separating the extract from the purified product;
separating the portion of the impurity compound from the extract;
preventing the formation of foam while separating the portion of the impurity compound from the extract using the defoamer;
forming an impurities stream and a regenerated solvent; and
recycling at least a portion of the regenerated solvent to contact the inlet stream.

2. The method of claim 1, wherein the solvent comprises an aqueous fluid.

3. The method of claim 1, wherein the extracting agent is configured to increase the solubility of the impurity compound in the solvent.

4. The method of claim 3, wherein the extracting agent comprises sodium hydrogen sulfite.

5. The method of claim 3, wherein the extracting agent comprises a compound selected from the group consisting of: hydrosulfite ion, sulfite ion, hydrazine, hydroxylamine, semicarbazide, phenylhydrazine, phenylhydroxylamine, salts thereof, aqueous solutions thereof, and any combination thereof.

6. The method of claim 3, further comprising reacting the extracting agent and the impurity compound to form an adduct in response to contacting the inlet stream with the solvent.

7. The method of claim 6, wherein separating the portion of the impurity compound from the extract comprises dissociating the adduct.

8. The method of claim 1, wherein the impurity compound comprises at least one of a ketone, an aldehyde, or any combination thereof.

9. The method of claim 1, wherein contacting the inlet stream with the solvent comprises at least one of contacting the inlet stream with the solvent in a counter-current flow, or contacting the inlet stream with the solvent in a cross-current flow.

10. The method of claim 1, further comprising:
feeding a feed stream comprising ethanol to one or more reactors;
contacting the ethanol with a catalyst in the one or more reactors;
dehydrogenating ethanol over the catalyst;
producing ethyl acetate and the impurity compound during the dehydrogenation to produce an effluent stream comprising the ethyl acetate and the impurity compound;
removing hydrogen from the effluent stream; and
providing the effluent stream as the inlet stream.

11. The method of claim 1, wherein the defoamer comprises a primary alcohol and an alcohol alkoxylate.

12. The method of claim 11, wherein the primary alcohol comprises one or more C₁₄ to C₃₂ primary linear alcohols.

13. The method of claim 11, wherein the alcohol alkoxylate comprises a hydrophobic group and a hydrophilic group.

14. The method of claim 11, where the defoamer comprises a silicone based defoamer.

15. A system for producing high purity ethyl acetate from ethanol, the system comprising:
a heater, wherein the heater is configured to heat an inlet ethanol stream;
at least one reactor comprising a dehydrogenation catalyst, an ethanol inlet configured to pass ethanol in the inlet ethanol stream from the heater over the dehydrogenation catalyst to produce a product stream comprising ethyl acetate and impurities;
an extraction unit configured to receive the product stream from the at least one reactor, contact a liquid solvent feed stream with the product stream, provide an extract stream comprising a portion of the impurities in the product stream, and provide a purified product stream, wherein the liquid solvent feed stream comprises an extracting agent and a defoamer; and
a stripping unit configured to receive the extract stream from the extraction unit, separate the impurities from the extract stream, provide an outlet impurities stream, and provide a regenerated solvent stream back to the extraction unit as at least a portion of the liquid solvent feed stream.

16. The system of claim 15, further comprising a drying unit, wherein the drying unit is configured to receive the purified product stream from the extraction unit, remove at least a portion of any water in the purified product stream, and provide a dried purified product stream.

17. The system of claim 15, wherein the extraction unit comprises a liquid-liquid contact vessel.

18. The system of claim 15, wherein the stripping unit comprises a distillation column.

19. A method of producing ethyl acetate, the method comprising:
feeding a feed stream comprising ethanol to one or more reactors;
contacting the ethanol with a catalyst in the one or more reactors;
producing ethyl acetate and one or more byproducts during the dehydrogenation to produce an effluent stream comprising the ethyl acetate and the one or more byproducts;
separating a portion of the one or more byproducts to produce a purified product stream, wherein the solvent comprises an extracting agent and a defoamer; and
separating at least a portion of the byproducts from the purified product stream using one or more distillation columns.

20. The method of claim 19, wherein separating the portion of the one or more byproducts to produce the purified product stream comprises using liquid-liquid extraction in the presence of a solvent to produce the purified product stream.

21. The method of claim 19, wherein the byproducts comprise a first portion having boiling points above the boiling point of ethyl acetate and a second portion having boiling points below the boiling point of ethyl acetate, and wherein separating at least the portion of the byproducts from the purified product stream comprises:
separating the first portion of the byproducts in a first distillation column to produce a first overhead stream comprising a majority of the first portion of the byproducts, and a first bottoms stream comprising a majority of the ethyl acetate and the second portion of the byproducts; and
separating the second portion of the byproducts from the first bottoms stream in a second distillation column to produce a second overhead stream comprising a majority of the ethyl acetate and a second bottoms stream comprising a majority of the second portion of the byproducts.

22. The method of claim 21, wherein the second overhead stream comprises at least 95 wt. % of a mass flowrate of the ethyl acetate in the purified product stream.

23. The method of claim 19, wherein the byproducts comprise a first portion having boiling points above the boiling point of ethyl acetate and a second portion having boiling points below the boiling point of ethyl acetate, and wherein separating at least the portion of the byproducts from the purified product stream comprises:
separating the second portion of the byproducts in a first distillation column to produce a first overhead stream comprising a majority of the first portion of the byproducts and the ethyl acetate, and a first bottoms stream comprising a majority of the second portion of the byproducts; and
separating the first portion of the byproducts from the first overhead stream in a second distillation column to produce a second overhead stream comprising a majority of the first portion of the byproducts and a second bottoms stream comprising a majority of the ethyl acetate.

24. The method of claim 23, wherein the second bottoms stream comprises at least 95 wt. % of a mass flowrate of the ethyl acetate in the purified product stream.
